(19) United States
(12) Patent Application Publication
(10) Pub. No.: US 2012/0003151 A1
(43) Pub. Date: Jan. 5, 2012

(54) METHOD FOR EARLY IMAGING OF ATHEROSCLEROSIS

(75) Inventors: Philip Stewart Low, West Lafayette, IN (US); Wilfredo Ayala-Lopez, Las Piedras, PR (US)

(73) Assignee: PURDUE RESEARCH FOUNDATION, West Lafayette, IN (US)

(21) Appl. No.: 13/254,637

(22) PCT Filed: Mar. 5, 2010
(86) PCT No.: PCT/US10/26406

§ 371 Date: Sep. 2, 2011

Related U.S. Application Data
(60) Provisional application No. 61/157,847, filed on Mar. 5, 2009.
(60) Provisional application No. 61/235,220, filed on Aug. 19, 2009.

(51) Int. Cl. A61K 51/04; A61K 49/00

(52) U.S. Cl. 424/1.65; 424/9.1; 424/9.6

(57) ABSTRACT

The invention relates to methods of detecting active atherosclerotic plaques associated with blood vessel walls wherein the plaques comprise activated macrophages having accessible binding sites for a ligand. In one embodiment, plaques that block from about 2% to about 60% of the lumen of a blood vessel can be detected.

### CROSS REFERENCE TO RELATED APPLICATIONS

This application claims priority under 35 U.S.C. §119(e) to U.S. Provisional Application No. 61/157,847, filed Mar. 5, 2009 and U.S. Provisional Application No. 61/235,220, filed Aug. 19, 2009, which are expressly incorporated by reference herein.

### FIELD OF THE INVENTION

This invention relates to a method for detecting active atherosclerotic plaques. More particularly, ligands that bind to activated macrophages are conjugated to a chromophore or to a chemical moiety capable of emitting radiation for administration to a diseased host for detecting active atherosclerotic plaques.

### BACKGROUND AND SUMMARY OF THE INVENTION

Activated macrophages can participate in the immune response by nonspecifically engulfing and killing foreign pathogens within the macrophage, by displaying degraded peptides from foreign proteins on the macrophage cell surface where they can be recognized by other immune cells, and by secreting cytokines and other factors that modulate the function of T and B lymphocytes, resulting in further stimulation of immune responses. Activated macrophages can also contribute to the pathophysiology of disease in some instances. For example, activated macrophages can contribute to atherosclerosis, rheumatoid arthritis, autoimmune disease states, and graft versus host disease.

Atherosclerosis is initiated when a fatty streak forms within a blood vessel wall. Formation of fatty streaks is believed to result from accumulation of lipoprotein particles in the intima layer of the blood vessel wall, the layer of the vessel wall underlying the luminal endothelial cell layer. Lipoprotein particles can associate with extracellular matrix components in the intima layer and can become inaccessible to plasma antioxidants, resulting in oxidative modification of the lipoprotein particles. Such oxidative modification may trigger a local inflammatory response resulting in adhesion of activated macrophages and T lymphocytes to the luminal endothelium followed by migration into the intima layer. The oxidized lipoprotein particles themselves can act as chemoattractants for cells of the immune system, such as macrophages and T cells, or can induce cells in the vascular wall to produce chemoattractants. The atherosclerotic lesion then forms a fibrous cap with a lipid-rich core filled with activated macrophages. Atherosclerotic lesions that are unstable are characterized by local inflammation, and lesions that have ruptured and have caused fatal myocardial infarction are characterized by an infiltration of activated macrophages and T lymphocytes.

The present invention relates to a method of detecting active atherosclerotic plaques in blood vessel walls. In accordance with the invention a ligand, that binds to a receptor which is preferentially expressed/presented on the surface of activated macrophages relative to resting macrophages, is conjugated to a chromophore or a chemical moiety capable of emitting radiation and the ligand conjugates are administered to a patient being evaluated for atherosclerosis. The ligand conjugates bind to activated macrophages associated with active atherosclerotic plaques and emit light (i.e., ligand-chromophore conjugates) or radiation (i.e., ligand-chemical moiety conjugates) and can be detected using a catheter-based device or by external imaging, such as by using X-ray detection. Accordingly, the ligand conjugates can be used to distinguish active atherosclerotic plaques containing activated macrophages from inactive plaques.

Because many unstable (i.e., active) atherosclerotic plaques, capable of rupturing and causing acute atherosclerotic syndromes do not produce significant luminal narrowing of blood vessels, particularly in the coronary circulation, the method of the present invention represents a significant advance in diagnosing the risk of myocardial infarction, and in evaluating the need for clinical intervention, in patients suffering from atherosclerosis.

In one embodiment of the invention, a method of detecting active atherosclerotic plaques wherein the plaques comprise activated macrophages having accessible binding sites for a ligand, and wherein the plaques block from about 2% to about 20% of the lumen of a blood vessel, said method comprising the steps of:

administering to a patient being evaluated for atherosclerosis an effective amount of a composition comprising a conjugate of the general formula

L-X

wherein the group L comprises the ligand and wherein the ligand is a folate, and the group X comprises a chromophore capable of emitting light under predetermined conditions;

allowing sufficient time for the ligand conjugate to bind to activated macrophages associated with the active plaques;

subjecting the blood vessel walls to the predetermined conditions; and

detecting active plaques by detecting light emitted by the chromophore using a catheter-based device or by external imaging, wherein the plaques block from about 2% to about 20% of the lumen of a blood vessel is described.

In another embodiment, a method of detecting active atherosclerotic plaques associated with blood vessel walls wherein the plaques comprise activated macrophages having accessible binding sites for a ligand, and wherein the plaques block from about 2% to about 20% of the lumen of a blood vessel, said method comprising the steps of:

administering to a patient suffering from atherosclerosis an effective amount of a composition comprising a conjugate of the general formula

L-X

wherein the group L comprises the ligand and wherein the ligand is a folate, and the group X comprises a chemical moiety capable of emitting radiation;

allowing sufficient time for the ligand conjugate to bind to the activated macrophages associated with the active plaques; and

detecting active plaques by detecting radiation emitted by the chemical moiety using a catheter-based device or by external imaging, wherein the plaques block from about 2% to about 20% of the lumen of a blood vessel is described.

In another embodiment, a pharmaceutical composition for detecting active atherosclerotic plaques wherein the plaques comprise activated macrophages having accessible binding sites for a ligand, and wherein the plaques block from about 4% to about 20% of the lumen of a blood vessel comprising an effective amount of the conjugate of the formula

L-X

wherein the group L comprises the ligand and wherein the ligand is a folate, and the group X comprises a chromophore capable of emitting light under predetermined conditions is described.

In another embodiment, a pharmaceutical composition for detecting active atherosclerotic plaques wherein the plaques comprise activated macrophages having accessible binding sites for a ligand, and wherein the plaques block from about 2% to about 20% of the lumen of a blood vessel comprising an effective amount of a conjugate of the general formula

L-X

wherein the group L comprises the ligand and wherein the ligand is a folate, and the group X comprises a chemical moiety capable of emitting radiation is described.

### BRIEF DESCRIPTION OF THE DRAWINGS

FIG. 1 shows EC20 imaging of ApoE−/− mice fed the Western Diet: Panel A shows mice fed the Western Diet for 10 weeks; Panel B shows mice fed the Western Diet for 25 weeks; and Panel C shows mice fed the Western Diet for 1 week.

FIG. 2 shows EC20 imaging of ApoE−/− mice fed the Western Diet for 0, 2, 12, and 26 weeks. Panel A shows ROI analysis of the EC20 signal in ApoE−/− mice. Panel B shows a graphical representation of the EC20 signal in ApoE−/− mice fed the Western Diet for 0, 2, 12, and 26 weeks.

FIG. 3 shows EC20 imaging of ApoE−/− mice fed the Western Diet for 0, 2, 12, and 25 weeks.

FIG. 4 shows hematoxylin and eosin (H&E) staining of atheromas versus time on the Western Diet. Panels A and C show H&E staining of the aortas of mice fed the Normal Diet. Panels B and D show H&E staining of the aortas of mice fed the Western Diet for 2 weeks. Panels A and B show the aortic arch. Panels C and D show the aortic root.

FIG. 5 shows hematoxylin and eosin staining of atheromas versus time on the Western Diet. Panels A and C show H&E staining of the aortas of mice fed the Western Diet for 12 weeks. Panels B and D show H&E staining of the aortas of mice fed the Western Diet for 26 weeks. Panels A and B show the aortic arch. Panels C and D show the aortic root.

FIG. 6 shows percent occlusion of the aortic lumen by atheromas. Panel A shows a table representing % occlusion at 0, 2, 12, and 26 weeks for ApoE−/− mice fed the Western Diet. Panel B is a graphical representation of the % occlusion of the aortic arch at 0, 2, 12, and 26 weeks. Panel C is a graphical representation of the % occlusion of the aortic root at 0, 2, 12, and 26 weeks.

FIG. 7 shows that EC20-^{99m}Tc targets the aortas of apoE−/− mice and can be used as an imaging agent for atherosclerosis. ApoE−/− mice were fed either a normal or Western diet for 25 weeks and then injected i.p. with either EC20-^{99m}Tc or EC20-^{99m}Tc+100-fold excess free folic acid. Radioimages were obtained on a Kodak Imaging Station (two animals shown in panel A; n=10), and regions-of-interest were quantitatively analyzed using instrument software (panel B; n=10). Mice were then euthanized and excised aortas were analyzed for radioactivity by γ-counting (panel C; n=5). When imaging, 5 mm lead shields were used to cover the abdomens to avoid interference from signals resulting from EC20-^{99m}Tc uptake in kidneys and bladder. Data in panel C are presented as means ±SD. *'**'^{#} p-values <0.05.

FIG. 8 shows that EC20-^{99m}Tc targets the aortic root and arch of apoE−/− mice. apoE−/− mice fed a normal or Western diet for a period of 25 weeks were injected with EC20-^{99m}Tc and thoracic aortas excised after allowing 4 hours for clearance of the radiopharmaceutical from folate receptor negative tissues. The aortas were exposed to a phosphor screen and images developed using a phosphorimager. (Upper panel) Aortas of mice on a normal diet; (middle panel) aortas of mice on a Western diet; (lower panel) aortas of mice on a Western diet but pre-injected with a 100-fold dose of free folic prior to the injection of EC20-^{99m}Tc.

FIG. 9 shows that treatment of apoE−/− mice on a Western diet with clodronate liposomes diminishes the uptake of EC20-^{99m}Tc. ApoE−/− mice on a Western diet for 8 weeks were treated for five days with single injections of PBS- or clodronate-liposomes (4 mg clodronate/injection) i.p. One day later EC20-^{99m}Tc was injected i.p. and animals were imaged 4 h later to assess cardiovascular uptake of the radiopharmaceutical (two animals shown in panel A). Regions-of-interest were then quantitatively analyzed using instrument software (panel B; n=5). In all cases, 5 mm lead shields were used to cover the abdomen to avoid any interference from signals resulting EC20-^{99m}Tc uptake in kidneys and bladder. Data are presented as means ±SD. * p<0.05.

FIG. 10 shows that EC20-^{99m}Tc preferentially accumulates in areas of high macrophage content within atherosclerotic plaques of apoE−/− mice. ApoE−/− mice on a Western diet for 25 weeks were injected with EC20-^{99m}Tc. After a 4 h tissue clearance period, aortas were dissected and embedded in OCT medium. Sections of the ascending aorta (panel A) and brachiocephalic artery (panel B) were prepared and exposed to a phosphor screen for 18 hours. Images were taken using a phosphorimager. Three consecutive sections were used for H&E staining, Mac-3 immunohistochemistry, or autoradiography on the phosphorimager. Bar=100 μm.

FIG. 11 shows percentage increase in FR+ macrophage numbers in apoE−/− mice on a Western diet. ApoE−/− mice were fed a normal (upper panels) or Western diet (lower panels) for a period of 25 weeks. Mice were euthanized and thoracic aortas excised and digested with collagenase and elastase. The resulting cell suspensions were analyzed by flow cytometry after incubation with Tri-color conjugated F4/80 antibody (macrophage marker) and rabbit anti-FR primary antibody followed by FITC-conjugated anti-rabbit IgG secondary antibody.

### DETAILED DESCRIPTION OF THE INVENTION

In one embodiment, a method of detecting active atherosclerotic plaques wherein the plaques comprise activated macrophages having accessible binding sites for a ligand, and wherein the plaques block from about 2% to about 20% of the lumen of a blood vessel is described. The method comprises the steps of administering to a patient being evaluated for atherosclerosis an effective amount of a composition comprising a conjugate of the general formula

L-X

wherein the group L comprises the ligand and wherein the ligand is folate, and the group X comprises a chromophore capable of emitting light under predetermined conditions; allowing sufficient time for the ligand conjugate to bind to activated macrophages associated with the active plaques; subjecting the blood vessel walls to the predetermined conditions using a catheter-based device or by external imaging; and detecting active plaques by detecting light emitted by the chromophore using a catheter-based device or by external imaging, wherein the plaques block from about 2% to about 20% of the lumen of a blood vessel.

In another embodiment, the method of any one of the preceding embodiments wherein the chromophore is selected from the group consisting of a fluorophore, a Raman enhancing dye, an hematoporphyrin, and derivatives thereof is described.

In another embodiment, the method of any one of the preceding embodiments wherein the chromophore is a fluorophore is described.

In another embodiment, the method of any one of the preceding embodiments wherein the fluorophore is selected from the group consisting of a fluorescein, a rhodamine, a cyanine, a DyLight Fluor, and an Alexa Fluor is described.

In yet another embodiment, the method of any one of the preceding embodiments wherein the fluorophore has the formula

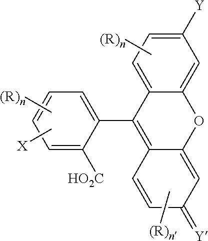

where X is oxygen, nitrogen, sulfur, S(O)₂, or C(O), and where X is attached via a divalent linker to the ligand; Y is OR^{a}, NR^{a}₂, or NR^{a}₃⁺; and Y′ is O, NR^{a}, or NR^{a}₂⁺; n is in each instance independently selected from 0, 1, 2, or 3; where each R is independently selected in each instance from H, alkyl, alkyloxy, heteroalkyl, fluoro, sulfonic acid, sulfonate, and salts thereof; and R^{a} is hydrogen, alkly, alkylsulfonic acid, or alkylsulfonate, and salts thereof; or at least one of R and Ra the atoms to which they are attached form a heterocycle is described.

In another embodiment, the method of any one of the preceding embodiments wherein the fluorophore has the formula

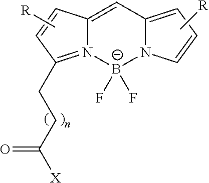

where X is oxygen, nitrogen, or sulfur, and where X is attached via a divalent linker to the ligand; and each R is independently selected in each instance from hydrogen, alkyl, heteroalkyl; and n is an integer from 0 to about 4 is described.

In another embodiment, the method of any one of the preceding embodiments wherein the fluorophore has the formula

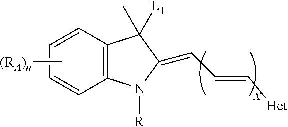

wherein R_{A} and R_{B} are independently selected in each instance from alkyl, heteroalkyl, alkylsulfonic acid, alkylsulfonate, or a salt thereof, or an amine or a derivative thereof; L₁ is an alkylene linked via a divalent linker to the ligand; R is independently selected in each instance from alkyl, heteroalkyl, or alkylsulfonic acid, or alkylsulfonate, or a salt thereof; n is independently in each instance an integer from 0 to about 3; x is an integer from about 1 to about 4; and Het is selected from the group consisting of

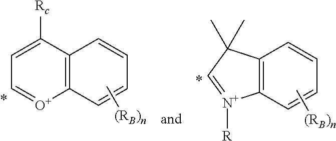

wherein * is the attachment point; and R_{C} is alkyl or heteroalkyl is described.

In another embodiment, the method of any one of the preceding embodiments wherein the fluorophore is selected from the group consisting of Cy3, Cy5, Cy7, Oregon Green 488, Oregon Green 514, AlexaFluor 488, AlexaFluor 647, tetramethylrhodamine, DyLight 680, CW 800, and Texas Red is described. In another embodiment, the method of any one of the preceding embodiments wherein the fluorophore is fluorescein is described.

In another embodiment, the method of any one of the preceding embodiments wherein the plaques block from about 2% to about 15% of the lumen of a blood vessel is described. In another embodiment, the method of any one of the preceding embodiments wherein the plaques block from about 2% to about 10% of the lumen of a blood vessel is described. In another embodiment, the method of any one of the preceding embodiments wherein the plaques block from about 4% to about 20% of the lumen of a blood vessel is described.

In another embodiment, the method of any one of the preceding embodiments wherein the folate has the formula

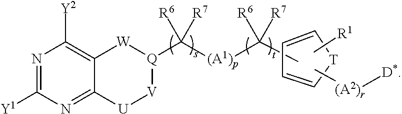

wherein Y¹ and Y² are each-independently selected from the group consisting of halo, R², OR², SR³, and NR⁴R⁵;

U, V, and W represent divalent moieties each independently selected from the group consisting of —(R^{6a})C═, —N═, —(R^{6a})C(R^{7a})—, and —N(R^{4a})—; Q is selected from the group consisting of C and CH; T is selected from the group consisting of S, O, N, and —C═C—;

A¹ and A² are each independently selected from the group consisting of oxygen, sulfur, —C(Z)-, —C(Z)O—, —OC(Z)-, —N(R^{4b})—, —C(Z)N(R^{4b})—, —N(R^{4b})C(Z)-, —OC(Z)N(R^{4b})—, —N(R^{4b})C(Z)O—, —N(R^{4b})C(Z)N(R^{5b})—, —S(O)—, —S(O)₂—, —N(R^{4a})S(O)₂—, —C(R^{6b})(R^{7b})—, —N(CδCH)—, —N(CH₂C≡CH)—, C₁-C₁₂ alkylene, and C₁-C₁₂ alkyeneoxy, where Z is oxygen or sulfur;

R¹ is selected-from the group consisting of hydrogen, halo, C₁-C₁₂ alkyl, and C₁-C₁₂ alkoxy; R², R³, R⁴, R^{4a}, R^{4b}, R⁵, R^{5b}, R^{6b}, and R^{7b} are each independently selected from the group consisting of hydrogen, halo, C₁-C₁₂ alkyl, C₁-C₁₂ alkoxy, C₁-C₁₂ alkanoyl, C₁-C₁₂ alkenyl, C₁-C₁₂ alkynyl, (C₁-C₁₂ alkoxy)carbonyl, and (C₁-C₁₂ alkylamino)carbonyl;

R⁶ and R⁷ are each independently selected from the group consisting of hydrogen, halo, C₁-C₁₂ alkyl, and C₁-C₁₂ alkoxy; or, R⁶ and R⁷ are taken together to form a carbonyl group; R^{6a} and R^{7a} are each independently selected from the group consisting of hydrogen, halo, C₁-C₁₂ alkyl, and C₁-C₁₂ alkoxy; or R^{6a} and R^{7a} are taken together to form a carbonyl group;

D is a divalent linker;

* represents the attachment point for X; and

n, p, r, s and t are each independently either 0 or 1 is described.

In another embodiment, the method of any one of the preceding embodiments wherein the folate has the formula

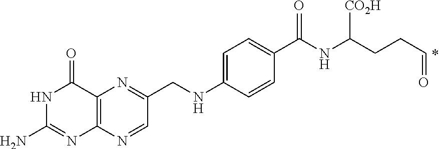

wherein * indicates the attachment point to the divalent linker attached to the chromaphore is described.

In another embodiment, a method of detecting active atherosclerotic plaques associated with blood vessel walls wherein the plaques comprise activated macrophages having accessible binding sites for a ligand, and wherein the plaques block from about 2% to about 20% of the lumen of a blood vessel is described. The method comprises the steps of administering to a patient suffering from atherosclerosis an effective amount of a composition comprising a conjugate of the general formula

L-X

wherein the group L comprises the ligand and wherein the ligand is folate, and the group X comprises a chemical moiety capable of emitting radiation; allowing sufficient time for the ligand conjugate to bind to the activated macrophages associated with the active plaques; and detecting active plaques by detecting radiation emitted by the chemical moiety using a catheter-based device or by external imaging, wherein the plaques block from about 2% to about 20% of the lumen of a blood vessel.

In another embodiment, the preceding embodiment wherein the chemical moiety comprises a metal chelating moiety is described. In another embodiment, the method of any one of the preceding embodiments wherein the chemical moiety further comprises a metal cation is described. In another embodiment, the method of any one of the preceding embodiments wherein the metal cation is a radionuclide is described. In another embodiment, the method of any one of the preceding embodiments wherein the radionuclide is ^{99m}Tc is described.

In another embodiment, the method of any one of the preceding embodiments wherein the metal cation is a nuclear magnetic resonance imaging enhancing agent is described.

In another embodiment, the method of any one of the preceding embodiments wherein the folate has the formula

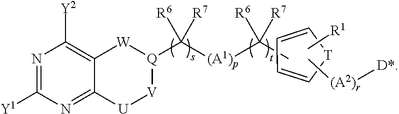

wherein Y¹ and Y² are each-independently selected from the group consisting of halo, R², OR², SR³, and NR⁴R⁵;

U, V, and W represent divalent moieties each independently selected from the group consisting of —(R^{6a})C═, —N═, —(R^{6a})C(R^{7a})—, and —N(R^{4a})—; Q is selected from the group consisting of C and CH; T is selected from the group consisting of S, O, N, and —C═C—;

A¹ and A² are each independently selected from the group consisting of oxygen, sulfur, —C(Z)-, —C(Z)O—, —OC(Z)-, —N(R^{4b})—, —C(Z)N(R^{4b})—, —N(R^{4b})C(Z)-, —OC(Z)N(R^{4b})—, —N(R^{4b})C(Z)O—, —N(R^{4b})C(Z)N(R^{5b})—, —S(O)—, —S(O)₂—, —N(R^{4a})S(O)₂—, —C(R^{6b})(R^{7b})—, —N(C≡CH)—, —N(CH₂C≡CH)—, C₁-C₁₂ alkylene, and C₁-C₁₂ alkyeneoxy, where Z is oxygen or sulfur;

R¹ is selected-from the group consisting of hydrogen, halo, C₁-C₁₂ alkyl, and C₁-C₁₂ alkoxy; R², R³, R⁴, R^{4a}, R^{4b}, R⁵, R^{5b}, R^{6b}, and R^{7b} are each independently selected from the group consisting of hydrogen, halo, C₁-C₁₂ alkyl, C₁-C₁₂ alkoxy, C₁-C₁₂ alkanoyl, C₁-C₁₂ alkenyl, C₁-C₁₂ alkynyl, (C₁-C₁₂ alkoxy)carbonyl, and (C₁-C₁₂ alkylamino)carbonyl;

R⁶ and R⁷ are each independently selected from the group consisting of hydrogen, halo, C₁-C₁₂ alkyl, and C₁-C₁₂ alkoxy; or, R⁶ and R⁷ are taken together to form a carbonyl group; R^{6a} and R^{7a} are each independently selected from the group consisting of hydrogen, halo, C₁-C₁₂ alkyl, and C₁-C₁₂ alkoxy; or R^{6a} and R^{7a} are taken together to form a carbonyl group;

D is a divalent linker;

* represents the attachment point for X; and

n, p, r, s and t are each independently either 0 or 1 is described.

In another embodiment, the method of any one of the preceding embodiments wherein the conjugate comprises a compound of the formula

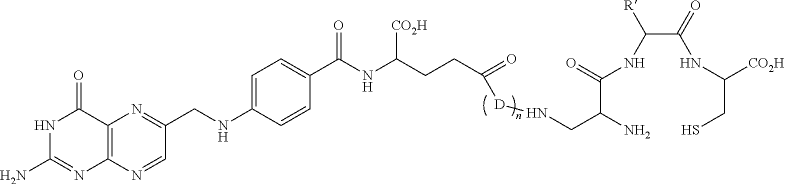

wherein R′ is hydrogen, or R′ selected from the group consisting of alkyl, aminoalkyl, carboxyalkyl, hydroxyalkyl, heteroalkyl, aryl, arylalkyl and heteroarylalkyl, each of which is optionally substituted; D is a divalent linker, n is 0 or 1 is described.

In another embodiment, the method of any one of the preceding embodiments wherein the conjugate has the formula

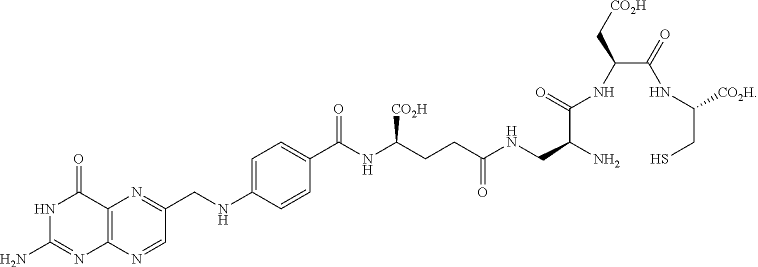

In another embodiment, the method of any one of the preceding embodiments wherein the plaques block from about 4% to about 10% of the lumen of a blood vessel.

In another embodiment, the method of any one of the preceding embodiments wherein the plaques block from about 4% to about 15% of the lumen of a blood vessel.

In another embodiment, the method of any one of the preceding embodiments wherein the plaques block from about 4% to about 20% of the lumen of a blood vessel.

In another embodiment, described herein is pharmaceutical composition for detecting active atherosclerotic plaques wherein the plaques comprise activated macrophages having accessible binding sites for a ligand, and wherein the plaques block from about 4% to about 20% of the lumen of a blood vessel comprising an effective amount of the conjugate of the formula

L-X

wherein the group L comprises the ligand and wherein the ligand is a folate, and the group X comprises a chromophore capable of emitting light under predetermined conditions is described.

In another embodiment, the composition of the preceding embodiment wherein the chromophore is selected from the group consisting of a fluorophore, a Raman enhancing dye, an hematoporphyrin, and derivatives thereof is described.

In another embodiment, the composition of any one of the preceding embodiments wherein the chromophore is a fluorophore is described.

In another embodiment, the composition of any one of the preceding embodiments wherein the fluorophore is selected from the group consisting of a fluorescein, a rhodamine, a cyanine, a DyLight Fluor, and an Alexa Fluor is described.

In another embodiment, the composition of any one of the preceding embodiments wherein the chromophore has the formula

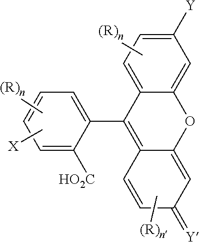

where X is oxygen, nitrogen, sulfur, S(O)₂, or C(O), and where X is attached via a divalent linker to the ligand; Y is OR^{a}, NR^{a}₂, or NR^{a}₃⁺; and Y′ is O, NR^{a}, or NR^{a}₂⁺; n is in each instance independently selected from 0, 1, 2, or 3; where each R is independently selected in each instance from H, alkyl, alkyloxy, heteroalkyl, fluoro, sulfonic acid, sulfonate, and salts thereof; and R^{a} is hydrogen, alkly, alkylsulfonic acid, or alkylsulfonate, and salts thereof; or at least one of R and Ra the atoms to which they are attached form a heterocycle is described.

In another embodiment, the composition of any one of the preceding embodiments wherein the chromophore has the formula

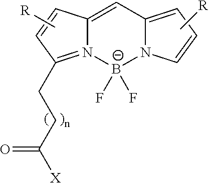

where X is oxygen, nitrogen, or sulfur, and where X is attached via a divalent linker to the ligand; and each R is independently selected in each instance from hydrogen, alkyl, heteroalkyl; and n is an integer from 0 to about 4 is described.

In another embodiment, the composition of any one of the preceding embodiments wherein the chromophore has the formula

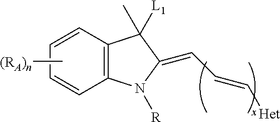

wherein R_{A} and R_{B} are independently selected in each instance from alkyl, heteroalkyl, alkylsulfonic acid, alkylsulfonate, or a salt thereof, or an amine or a derivative thereof; L₁ is an alkylene linked via a divalent linker to the ligand; R is independently selected in each instance from alkyl, heteroalkyl, or alkylsulfonic acid, or alkylsulfonate, or a salt thereof; n is independently in each instance an integer from 0 to about 3; x is an integer from about 1 to about 4; and Het is selected from the group consisting of

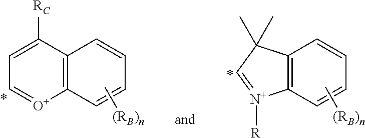

wherein * is the attachment point; and R_{C} is alkyl or heteroalkyl is described.

In another embodiment, the composition of any one of the preceding embodiments wherein the fluorphore is selected from the group consisting of Cy3, Cy5, Cy7, Oregon Green 488, Oregon Green 514, AlexaFluor 488, AlexaFluor 647, tetramethylrhodamine, DyLight 680, CW 800, and Texas Red is described is described.

In another embodiment, the composition of any one of the preceding embodiments wherein the fluorophore is fluorescein is described.

In another embodiment, the composition of any one of the preceding embodiments wherein the plaques block from about 4% to about 15% of the lumen of a blood vessel is described.

In another embodiment, the composition of any one of the preceding embodiments wherein the plaques block from about 4% to about 20% of the lumen of a blood vessel is described.

In another embodiment, the composition of any one of the preceding embodiments wherein the plaques block from about 4% to about 10% of the lumen of a blood vessel is described.

In another embodiment, the composition of any one of the preceding embodiments wherein the folate has the formula

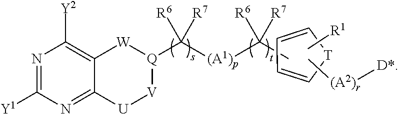

wherein Y¹ and Y² are each-independently selected from the group consisting of halo, R², OR², SR³, and NR⁴R⁵;

U, V, and W represent divalent moieties each independently selected from the group consisting of —(R^{6a})C═, —N═, —(R^{6a})C(R^{7a})—, and —N(R^{4a})—; Q is selected from the group consisting of C and CH; T is selected from the group consisting of S, O, N, and —C═C—;

A¹ and A² are each independently selected from the group consisting of oxygen, sulfur, —C(Z)-, —C(Z)O—, —OC(Z)-, —N(R^{4b})—, —C(Z)N(R^{4b})—, —N(R^{4b})C(Z)-, —OC(Z)N(R^{4b})—, —N(R^{4b})C(Z)O—, —N(R^{4b})C(Z)N(R^{5b})—, —S(O)—, —S(O)₂—, —N(R^{4a})S(O)₂—, —C(R^{6b})(R^{7b})—, —N(C≡CH)—, —N(CH₂C≡CH)—, C₁-C₁₂ alkylene, and C₁-C₁₂ alkyeneoxy, where Z is oxygen or sulfur;

R¹ is selected-from the group consisting of hydrogen, halo, C₁-C₁₂ alkyl, and C₁-C₁₂ alkoxy; R², R³, R⁴, R^{4a}, R^{4b}, R⁵, R^{5b}, R^{6b}, and R^{7b} are each independently selected from the group consisting of hydrogen, halo, C₁-C₁₂ alkyl, C₁-C₁₂ alkoxy, C₁-C₁₂ alkanoyl, C₁-C₁₂ alkenyl, C₁-C₁₂ alkynyl, (C₁-C₁₂ alkoxy)carbonyl, and (C₁-C₁₂ alkylamino)carbonyl;

R⁶ and R⁷ are each independently selected from the group consisting of hydrogen, halo, C₁-C₁₂ alkyl, and C₁-C₁₂ alkoxy; or, R⁶ and R⁷ are taken together to form a carbonyl group; R^{6a} and R^{7a} are each independently selected from the group consisting of hydrogen, halo, C₁-C₁₂ alkyl, and C₁-C₁₂ alkoxy; or R^{6a} and R^{7a} are taken together to form a carbonyl group;

D is a divalent linker;

* represents the attachment point for X; and

n, p, r, s and t are each independently either 0 or 1 is described.

In another embodiment, the composition of any one of the preceding embodiments wherein the folate has the formula

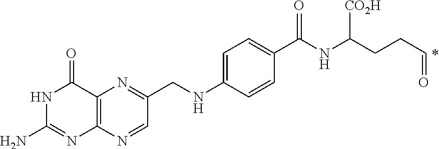

wherein * indicates the attachment point to the divalent linker attached to the chromophore is described.

In another embodiment, a pharmaceutical composition for detecting active atherosclerotic plaques wherein the plaques comprise activated macrophages having accessible binding sites for a ligand, and wherein the plaques block from about 2% to about 20% of the lumen of a blood vessel comprising an effective amount of a conjugate of the general formula

L-X

wherein the group L comprises the ligand and wherein the ligand is a folate, and

the group X comprises a chemical moiety capable of emitting radiation is described.

In another embodiment, the composition of the preceding embodiment wherein the chemical moiety comprises a metal chelating moiety is described.

In another embodiment, the composition of the preceding embodiment wherein the chemical moiety further comprises a metal cation is described.

In another embodiment, the composition of the preceding embodiment wherein the metal cation is a radionuclide is described.

In another embodiment, the composition of the preceding embodiment wherein the radionuclide is ^{99m}Tc is described.

In another embodiment, the composition of the preceding embodiment wherein the metal cation is a nuclear magnetic resonance imaging enhancing agent is described.

In another embodiment, the composition of any one of the preceding embodiments wherein the folate has the formula

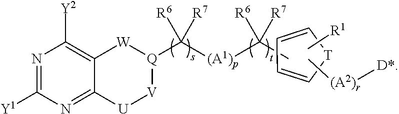

wherein Y¹ and Y² are each-independently selected from the group consisting of halo, R², OR², SR³, and NR⁴R⁵;

U, V, and W represent divalent moieties each independently selected from the group consisting of —(R^{6a})C═, —N═, —(R^{6a})C(R^{7a})—, and —N(R^{4a})—; Q is selected from the group consisting of C and CH; T is selected from the group consisting of S, O, N, and —C═C—;

A¹ and A² are each independently selected from the group consisting of oxygen, sulfur, —C(Z)-, —C(Z)O—, —OC(Z)-, —N(R^{4b})—, —C(Z)N(R^{4b})—, —N(R^{4b})C(Z)-, —OC(Z)N(R^{4b})—, —N(R^{4b})C(Z)O—, —N(R^{4b})C(Z)N(R^{5b})—, —S(O)—, —S(O)₂—, —N(R^{4a})S(O)₂—, —C(R^{6b})(R^{7b})—, —N(C≡CH)—, —N(CH₂C≡CH)—, C₁-C₁₂ alkylene, and C₁-C₁₂ alkyeneoxy, where Z is oxygen or sulfur;

R¹ is selected-from the group consisting of hydrogen, halo, C₁-C₁₂ alkyl, and C₁-C₁₂ alkoxy; R², R³, R⁴, R^{4a}, R^{4b}, R⁵, R^{5b}, R^{6b}, and R^{7b} are each independently selected from the group consisting of hydrogen, halo, C₁-C₁₂ alkyl, C₁-C₁₂ alkoxy, C₁-C₁₂ alkanoyl, C₁-C₁₂ alkenyl, C₁-C₁₂ alkynyl, (C₁-C₁₂ alkoxy)carbonyl, and (C₁-C₁₂ alkylamino)carbonyl;

R⁶ and R⁷ are each independently selected from the group consisting of hydrogen, halo, C₁-C₁₂ alkyl, and C₁-C₁₂ alkoxy; or, R⁶ and R⁷ are taken together to form a carbonyl group; R^{6a} and R^{7a} are each independently selected from the group consisting of hydrogen, halo, C₁-C₁₂ alkyl, and C₁-C₁₂ alkoxy; or R^{6a} and R^{7a} are taken together to form a carbonyl group;

D is a divalent linker;

* represents the attachment point for X; and

n, p, r, s and t are each independently either 0 or 1 is described.

In another embodiment, the any one of the preceding embodiment wherein the conjugate comprises a compound of the formula

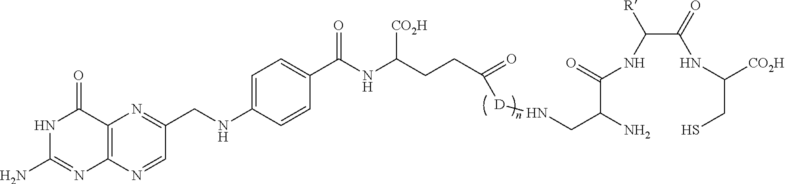

wherein R′ is hydrogen, or R′ selected from the group consisting of alkyl, aminoalkyl, carboxyalkyl, hydroxyalkyl, heteroalkyl, aryl, arylalkyl and heteroarylalkyl, each of which is optionally substituted; D is a divalent linker, n is 0 or 1 is described.

In another embodiment, the composition of any one of the preceding embodiments wherein the conjugate has the formula

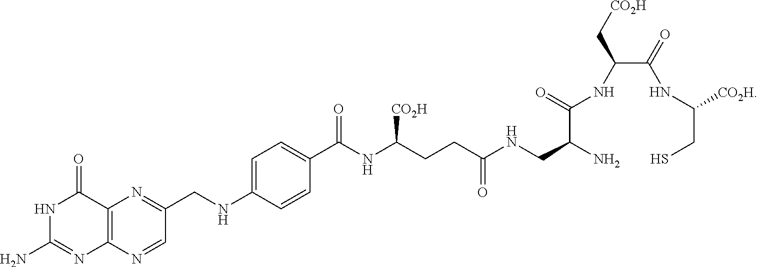

In another embodiment, the composition of any one of the preceding embodiments further comprising a carrier, diluent, excipient, or combination thereof is described.

In another embodiment, a kit comprising the composition of any one of the preceding composition embodiments in a sterile container is describedr.

In another embodiment, the kit of the preceding embodiment further comprising instructions for using the composition to detect active atherosclerotic plaques in a patient is described.

The ligand conjugates bind to activated macrophages associated with active atherosclerotic plaques. The light or radiation emitted by the ligand-chromophore conjugate or the chemical moiety, respectively, is detected using a catheter-based device or externally using such methods as X-ray detection.

As used herein, the word “detecting” refers to identifying atherosclerotic plaques or monitoring atherosclerotic plaques (e.g., identifying atherosclerotic plaques by detecting light or radiation emitted by a ligand-chromophore conjugate or a chemical moiety, respectively, using a catheter-based device or external imaging). The atherosclerotic plaques may be associated with blood vessel walls.

As used herein, “active atherosclerotic plaques” are plaques that contain activated macrophages having accessible binding sites for a ligand, e.g., a folate.

In accordance with the invention, the word “catheter” means any catheter, guidewire, or other device capable of transluminal delivery (i.e., delivery into the lumen of blood vessels) of optical energy or of radiation, and/or any catheter, guidewire, or other device capable of detecting, in the lumen of blood vessels, light or radioactivity emitted from the ligand conjugates used in accordance with the method of the present invention, and/or any catheter, guidewire, or other device capable of delivering a therapeutic drug to the lumen of blood vessels.

In accordance with the present invention, the ligand conjugates can be formed from a wide variety of ligands, including any ligand that binds to a receptor expressed or presented on the surface of activated macrophages that is not expressed/presented or is not present in significant amounts on the surface of resting macrophages. Such ligands include N-formyl peptides (e.g., f-Met-Leu-Phe), high mobility group factor 1 protein (HMGB1), hyaluronan fragments, HSP-70, toll-like receptor ligands, scavenger receptor ligands, co-receptors for antigen presentation, ligands that bind to the CD68, BER-MAG3, RFD7, CD4, CD14, and HLA-D markers on activated macrophages, ligands that bind to urokinase plasminogen activator receptors (e.g., the WX-360 peptide), antibodies, or fragments thereof, that bind preferentially to activated macrophages, and vitamins or receptor-binding vitamin analogs/derivatives. The ligand conjugates are capable of preferentially binding to activated macrophages compared to resting macrophages due to preferential expression of the receptor for the ligand on activated macrophages.

Acceptable vitamin moieties that can be used as ligands in accordance with the invention include niacin, pantothenic acid, folic acid, riboflavin, thiamine, biotin, vitamin B₁₂, and the lipid soluble vitamins A, D, E and K. These vitamins, and their receptor-binding analogs and derivatives, constitute the targeting entity that can be coupled with a chromophore or a chemical moiety, capable of emitting radiation, to form the ligand conjugates for use in accordance with the invention. Preferred vitamin moieties include folic acid, biotin, riboflavin, thiamine, vitamin B₁₂, and receptor-binding analogs and derivatives of these vitamin molecules, and other related vitamin receptor-binding molecules (see U.S. Pat. No. 5,688,488, incorporated herein by reference). Exemplary of a vitamin analog is a folate analog containing a glutamic acid residue in the D configuration (folic acid normally contains one glutamic acid in the L configuration linked to pteroic acid).

In the ligand conjugates of the general formula L-X in accordance with the present invention, the group L is a ligand capable of binding to activated macrophages as compared to resting macrophages as described above. In one embodiment the activated macrophage binding ligand is folic acid, a folic acid analog/derivative or other folate receptor binding molecules.

In other embodiments, the targeting ligand L is a folate, an analog of folate, or a derivative of folate. It is to be understood as used herein, that the term folate is used both individually and collectively to refer to folic acid itself, and/or to such analogs and derivatives of folic acid that are capable of binding to folate receptors.

Illustrative embodiments of folate analogs and/or derivatives include folinic acid, pteropolyglutamic acid, and folate receptor-binding pteridines such as tetrahydropterins, dihydrofolates, tetrahydrofolates, and their deaza and dideaza analogs. The terms “deaza” and “dideaza” analogs refer to the art-recognized analogs having a carbon atom substituted for one or two nitrogen atoms in the naturally occurring folic acid structure, or analog or derivative thereof. For example, the deaza analogs include the 1-deaza, 3-deaza, 5-deaza, 8-deaza, and 10-deaza analogs of folate. The dideaza analogs include, for example, 1,5-dideaza, 5,10-dideaza, 8,10-dideaza, and 5,8-dideaza analogs of folate. Other folates useful as complex forming ligands include the folate receptor-binding analogs aminopterin, amethopterin (methotrexate), N¹⁰-methylfolate, 2-deamino-hydroxyfolate, deaza analogs such as 1-deazamethopterin or 3-deazamethopterin, and 3′,5′-dichloro-4-amino-4-deoxy-N¹⁰-methylpteroylglutamic acid (dichloromethotrexate). The foregoing folic acid analogs and/or derivatives are conventionally termed folates, reflecting their ability to bind with folate-receptors.

Additional analogs of folic acid that bind to folic acid receptors are described in US Patent Application Publication Serial Nos. 2005/0227985 and 2004/0242582, the disclosures of which are incorporated herein by reference. Illustratively, such folate analogs have the general formula:

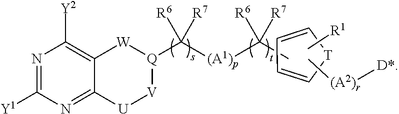

wherein Y¹ and Y² are each-independently selected from the group consisting of halo, R², OR², SR³, and NR⁴R⁵;

U, V, and W represent divalent moieties each independently selected from the group consisting of —(R^{6a})C═, —N═, —(R^{6a})C(R^{7a})—, and —N(R^{4a})—; Q is selected from the group consisting of C and CH; T is selected from the group consisting of S, O, N, and —C═C—;

A¹ and A² are each independently selected from the group consisting of oxygen, sulfur, —C(Z)-, —C(Z)O—, —OC(Z)-, —N(R^{4b})—, —C(Z)N(R^{4b})—, —N(R^{4b})C(Z)-, —OC(Z)N(R^{4b})—, —N(R^{4b})C(Z)O—, —N(R^{4b})C(Z)N(R^{5b})—, —S(O)—, —S(O)₂—, —N(R^{4a})S(O)₂—, —C(R^{6b})(R^{7b})—, —N(C≡CH)—, —N(CH₂C≡CH)—, C₁-C₁₂ alkylene, and C₁-C₁₂ alkyeneoxy, where Z is oxygen or sulfur;

R¹ is selected-from the group consisting of hydrogen, halo, C₁-C₁₂ alkyl, and C₁-C₁₂ alkoxy; R², R³, R⁴, R^{4a}, R^{4b}, R⁵, R^{5b}, R^{6b}, and R^{7b} are each independently selected from the group consisting of hydrogen, halo, C₁-C₁₂ alkyl, C₁-C₁₂ alkoxy, C₁-C₁₂ alkanoyl, C₁-C₁₂ alkenyl, C₁-C₁₂ alkynyl, (C₁-C₁₂ alkoxy)carbonyl, and (C₁-C₁₂ alkylamino)carbonyl;

R⁶ and R⁷ are each independently selected from the group consisting of hydrogen, halo, C₁-C₁₂ alkyl, and C₁-C₁₂ alkoxy; or, R⁶ and R⁷ are taken together to form a carbonyl group; R^{6a} and R^{7a} are each independently selected from the group consisting of hydrogen, halo, C₁-C₁₂ alkyl, and C₁-C₁₂ alkoxy; or R^{6a} and R^{7a} are taken together to form a carbonyl group;

D is a divalent linker;

* represents the attachment point for X; and

n, p, r, s and t are each independently either 0 or 1.

As used herein, it is to be understood that the term folate refers both individually to folic acid used in forming a conjugate, or alternatively to a folate analog or derivative thereof that is capable of binding to folate or folic acid receptors.

In another embodiment the activated macrophage binding ligand is a specific monoclonal or polyclonal antibody or Fab or scFv (i.e., a single chain variable region) fragments of an antibody capable of preferential binding to activated macrophages as compared to resting macrophages.

Activated macrophages express a 38 kD GPI-anchored folate receptor that binds folate and folate-derivatized compounds with subnanomolar affinity (i.e., <1 nM). Importantly, covalent conjugation of small molecules, proteins, and even liposomes to folic acid does not alter the vitamin's ability to bind the folate receptor. Because most cells use an unrelated reduced folate carrier to acquire the necessary folic acid, expression of the folate receptor is restricted to a few cell types. With the exception of kidney, choroid plexus, and placenta, normal tissues express low or nondetectable levels of the folate receptor. However, many malignant tissues, including ovarian, breast, bronchial, and brain cancers express significantly elevated levels of the receptor. Also, it has recently been reported that the folate receptor β, the nonepithelial isoform of the folate receptor, is expressed in active form on activated, but not resting synovial macrophages.

The binding site for the ligand can include receptors for any ligand molecule, or a derivative or analog thereof, capable of preferentially binding to a receptor uniquely expressed or preferentially expressed/presented on the surface of activated macrophages. A surface-presented protein uniquely expressed or preferentially expressed by activated macrophages is a receptor that is either not present or is present at insignificant concentrations on resting macrophages providing a means for preferential detection of activated macrophages. Accordingly, any receptor that is upregulated on activated macrophages compared to resting macrophages, or which is not expressed/presented on the surface of resting macrophages, or any receptor that is not expressed/presented on the surface of resting macrophages in significant amounts could be used for targeting. In one embodiment the site that binds the ligand conjugates used in accordance with the present invention is a vitamin receptor, for example, the folate receptor, which binds folate or an analog or derivative thereof.

In accordance with the invention the ligand conjugates can bind with high affinity to receptors on activated macrophages. The high affinity binding can be inherent to the ligand or the binding affinity can be enhanced by the use of a chemically modified ligand (i.e., an analog or a derivative) or by the particular chemical linkage, in the ligand conjugate, between the ligand and the chromophore or between the ligand and the chemical moiety capable of emitting radiation.

The chemical linkage in the ligand conjugate between the ligand and the chromophore or between the ligand and the chemical moiety can be a direct linkage or can be through an intermediary linker. If present, an intermediary linker can be any biocompatible linker known in the art. In one illustrative embodiment, the linker comprises about 1 to about 30 carbon atoms, in another illustrative embodiment, the linker comprises about 2 to about 20 carbon atoms. Lower molecular weight linkers (i.e., those having an approximate molecular weight of about 30 to about 300) are typically employed.

In one embodiment the linker comprises a heteroatom directly bonded to the ligand and the chromophore or to the ligand and the chemical moiety. In one embodiment the heteroatom is nitrogen. In another embodiment the linker comprises an optionally-substituted diaminoalkylene. In one embodiment the optionally-substituted diaminoalkylene is a diaminoacid. In another embodiment the linker comprises one or more optionally-substituted diaminoalkylene moieties, and one or more optionally-substituted amino acids. In one illustrative example the linker comprises glutamic acid.

In another illustrative embodiment, the linker includes one or more amino acids. In one variation, the linker includes a single amino acid. In another variation, the linker includes a peptide having from 2 to about 50, 2 to about 30, or 2 to about 20 amino acids. In another variation, the linker includes a peptide having from about 4 to about 8 amino acids. Such amino acids are illustratively selected from the naturally occurring amino acids, or stereoisomers thereof. The amino acid may also be any other amino acid, such as any amino acid having the general formula:

—N(R)—(CR′R″)_{q}—C(O)—

where R is hydrogen, alkyl, acyl, or a suitable nitrogen protecting group, R′ and R″ are hydrogen or a substituent, each of which is independently selected in each occurrence, and q is an integer such as 1, 2, 3, 4, or 5. Illustratively, R′ and/or R″ independently correspond to, but are not limited to, hydrogen or the side chains present on naturally occurring amino acids, such as methyl, benzyl, hydroxymethyl, thiomethyl, carboxyl, carboxylmethyl, guanidinopropyl, and the like, and derivatives and protected derivatives thereof. The above described formula includes all stereoisomeric variations. For example, the amino acid may be selected from asparagine, aspartic acid, cysteine, glutamic acid, lysine, glutamine, arginine, serine, ornithine, threonine, and the like. In one variation, the linker includes at least 2 amino acids selected from asparagine, aspartic acid, cysteine, glutamic acid, lysine, glutamine, arginine, serine, ornithine, and threonine. In another variation, the linker includes between 2 and about 5 amino acids selected from asparagine, aspartic acid, cysteine, glutamic acid, lysine, glutamine, arginine, serine, ornithine, and threonine. In another variation, the linker includes a tripeptide, tetrapeptide, pentapeptide, or hexapeptide consisting of amino acids selected from aspartic acid, cysteine, glutamic acid, lysine, arginine, and ornithine, and combinations thereof.

In another embodiment the linker may also include one or more spacer linkers. Illustrative spacer linkers are shown in the following table

The following non-limiting, illustrative spacer linkers are described where * indicates the point of attachment.

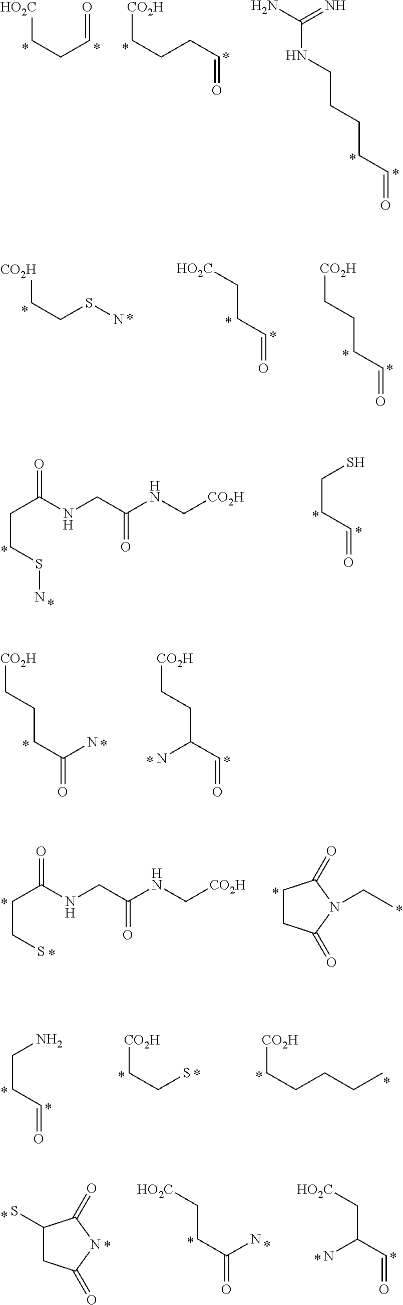

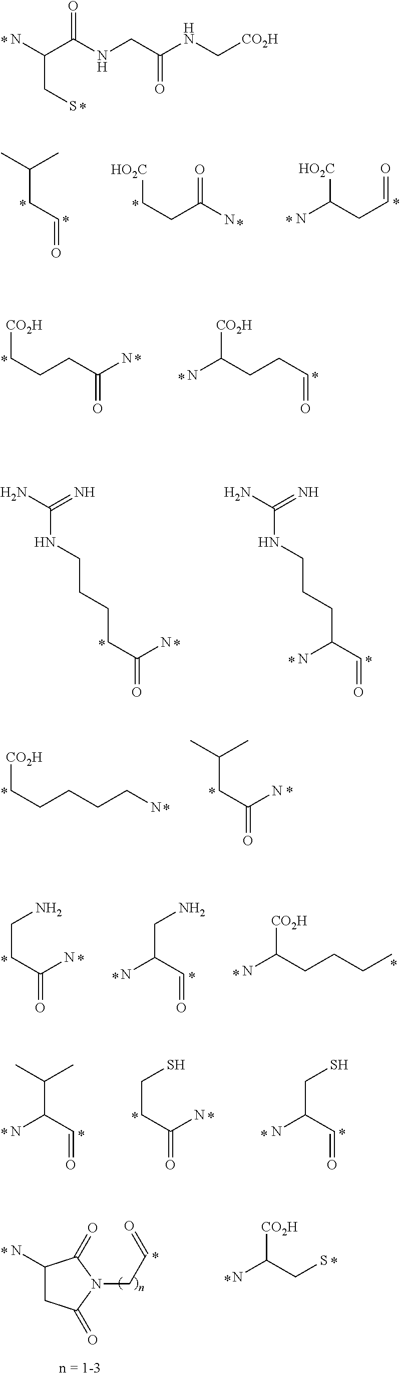

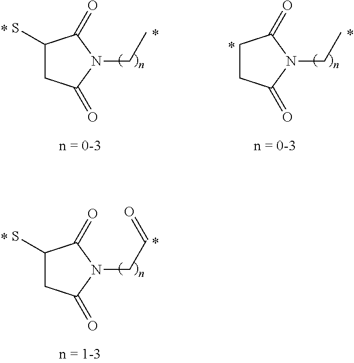

Generally, any manner of forming a complex between the ligand and the chromophore, between the ligand and the chemical moiety capable of emitting radiation, between a linker and the ligand, or between a linker and the chromophore or chemical moiety capable of emitting radiation can be utilized in accordance with the present invention. With or without a linker, the complex can be formed by conjugation of the components of the conjugate, for example, through hydrogen, ionic, or covalent bonds. Covalent bonding of the components of the conjugate can occur, for example, through the formation of amide, ester, disulfide, or imino bonds between acid, aldehyde, hydroxy, amino, sulfhydryl, or hydrazo groups. Also, in accordance with this invention a linker can comprise an indirect means for associating the ligand with the chromophore/chemical moiety, such as by connection through spacer arms or bridging molecules. Both direct and indirect means for association should not prevent the binding of the ligand to the receptor on the activated macrophages for operation of the method of the present invention. Alternatively, the ligand conjugate can be one comprising a liposome wherein the chemical moiety capable of emitting radiation, for example, is contained within a liposome which is itself covalently linked to the activated macrophage-binding ligand.

In the embodiment where the ligand is folic acid, an analog/derivative of folic acid, or any other folate receptor binding molecule, the folate ligand can be conjugated to the chromophore/chemical moiety by an art-recognized procedure that utilizes trifluoroacetic anhydride to prepare γ-esters of folic acid via a pteroyl azide intermediate. This procedure results in the synthesis of a folate ligand, conjugated to the chromophore/chemical moiety only through the γ-carboxy group of the glutamic acid groups of folate. Alternatively, folic acid analogs can be coupled by art-recognized procedures through the α-carboxy moiety of the glutamic acid group or both the α and γ carboxylic acid entities.

The amount of the conjugate effective for use in accordance with the method of the invention depends on many parameters, including the molecular weight of the conjugate, its route of administration, and its tissue distribution. In accordance with the invention an “effective amount” of the ligand conjugate is an amount sufficient to bind to activated macrophages and to be useful in the identification/monitoring of active atherosclerotic plaques. The effective amount of the ligand conjugate to be administered to a patient being evaluated for atherosclerosis can range from about 1 ng/kg to about 10 mg/kg, or from about 10 μg/kg to about 1 mg/kg, or from about 100 μg/kg to about 500 μg/kg.

The ligand conjugate can be administered in one or more doses (e.g., about 1 to about 3 doses) prior to the catheterization or external imaging procedure. The number of doses depends on the molecular weight of the conjugate, its route of administration, and its tissue distribution, among other factors. When used for identification/monitoring of active atherosclerotic plaques, the catheterization or external imaging procedure is typically performed about 1 to about 6 hours post-administration of the ligand conjugate targeted to activated macrophages, but the catheterization or external imaging procedure can be performed at any time post-administration of the ligand conjugate as long as binding of the ligand conjugate to activated macrophages is detectable.

The ligand conjugates administered in accordance with the method of this invention are preferably administered parenterally to the patient being evaluated for atherosclerosis, for example, intravenously, intradermally, subcutaneously, intramuscularly, or intraperitoneally, in combination with a pharmaceutically acceptable carrier. Suitable means for parenteral administration include needle (including microneedle) injectors, needle-free injectors and infusion techniques. Alternatively, the conjugates can be administered to the patient being evaluated for artherosclerosis by other medically useful procedures such as in an orally available formulation. In accordance with the invention, a “patient being evaluated for artherosclerosis” means any patient suspected of having artherosclerosis, whether symptomatic or not, who would benefit from an evaluation using the method of the present invention.

The conjugates used in accordance with this invention of the formula L-X are used in one aspect of this invention to formulate diagnostic compositions comprising diagnostically effective amounts of the conjugate and an acceptable carrier therefor. Examples of parenteral dosage forms include aqueous solutions of the conjugate, for example, a solution in isotonic saline, 5% glucose or other well-known pharmaceutically acceptable liquid carriers such as alcohols, glycols, esters and amides. The parenteral compositions for use in accordance with this invention can be in the form of a reconstitutable lyophilizate comprising the one or more doses of the ligand conjugate. Any orally available dosage forms known in the art can also be used.

In other embodiments of the compositions and methods described herein, pharmaceutically acceptable salts of the conjugates described herein are described. Pharmaceutically acceptable salts of the conjugates described herein include the acid addition and base salts thereof.

Suitable acid addition salts are formed from acids which form non-toxic salts. Illustrative examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts of the conjugates described herein are formed from bases which form non-toxic salts. Illustrative examples include the arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts.

In one embodiment, the conjugates described herein may be administered as a formulation in association with one or more pharmaceutically acceptable carriers. The carriers can be excipients. The choice of carrier will to a large extent depend on factors such as the particular mode of administration, the effect of the carrier on solubility and stability, and the nature of the dosage form. Pharmaceutical compositions suitable for the delivery of conjugates described herein and methods for their preparation will be readily apparent to those skilled in the art. Such compositions and methods for their preparation may be found, for example, in Remington: The Science & Practice of Pharmacy, 21th Edition (Lippincott Williams & Wilkins, 2005), incorporated herein by reference.

In some illustrative embodiments, formulations of ligand conjugates for diagnostic use for parenteral administration comprising: a) a pharmaceutically active amount of the ligand conjugate; b) a pharmaceutically acceptable pH buffering agent to provide a pH in the range of about pH 4.5 to about pH 9; c) an ionic strength modifying agent in the concentration range of about 0 to about 250 millimolar; or d) a water soluble viscosity modifying agent in the concentration range of about 0.5% to about 7% total formula weight; or any combinations of a), b), c) and d) are described.

In various illustrative embodiments, the pH buffering agents for use in the compositions and methods herein described are those agents known to the skilled artisan and include, for example, acetate, borate, carbonate, citrate, and phosphate buffers, as well as hydrochloric acid, sodium hydroxide, magnesium oxide, monopotassium phosphate, bicarbonate, ammonia, carbonic acid, hydrochloric acid, sodium citrate, citric acid, acetic acid, disodium hydrogen phosphate, borax, boric acid, sodium hydroxide, diethyl barbituric acid, and proteins, as well as various biological buffers, for example, TAPS, Bicine, Tris, Tricine, HEPES, TES, MOPS, PIPES, cacodylate, and MES.

In another illustrative embodiment, the ionic strength modulating agents include those agents known in the art, for example, glycerin, propylene glycol, mannitol, glucose, dextrose, sorbitol, sodium chloride, potassium chloride, and other electrolytes.

Useful viscosity modulating agents include but are not limited to, ionic and non-ionic water soluble polymers; crosslinked acrylic acid polymers such as the “carbomer” family of polymers, e.g., carboxypolyalkylenes that may be obtained commercially under the Carbopol® trademark; hydrophilic polymers such as polyethylene oxides, polyoxyethylene-polyoxypropylene copolymers, and polyvinylalcohol; cellulosic polymers and cellulosic polymer derivatives such as hydroxypropyl cellulose, hydroxyethyl cellulose, hydroxypropyl methylcellulose, hydroxypropyl methylcellulose phthalate, methyl cellulose, carboxymethyl cellulose, and etherified cellulose; gums such as tragacanth and xanthan gum; sodium alginate; gelatin, hyaluronic acid and salts thereof, chitosans, gellans or any combination thereof. It is preferred, but not required, that non-acidic viscosity enhancing agents, such as a neutral or basic agent be employed in order to facilitate achieving the desired pH of the formulation. If a uniform gel is desired, dispersing agents such as alcohol, sorbitol or glycerin can be added, or the gelling agent can be dispersed by trituration, mechanical mixing, or stirring, or combinations thereof. In one embodiment, the viscosity enhancing agent can also provide the base, discussed above. In one preferred embodiment, the viscosity modulating agent is cellulose that has been modified such as by etherification or esterification.

In one illustrative aspect, a pharmaceutically acceptable carrier includes any and all solvents, dispersion media, coatings, antibacterial and antifungal agents, isotonic and absorption delaying agents, and the like, and combinations thereof, that are physiologically compatible. In some embodiments, the carrier is suitable for parenteral administration. Pharmaceutically acceptable carriers include sterile aqueous solutions or dispersions and sterile powders for the extemporaneous preparation of sterile injectable solutions or dispersions. Supplementary active compounds can also be incorporated into compositions of the invention.

In various embodiments, liquid formulations may include suspensions and solutions. Such formulations may comprise a carrier, for example, water, ethanol, polyethylene glycol, propylene glycol, methylcellulose or a suitable oil, and one or more emulsifying agents and/or suspending agents. Liquid formulations may also be prepared by the reconstitution of a solid, for example, from a sachet.

In one embodiment, an aqueous suspension may contain the active materials in admixture with appropriate excipients. Such excipients are suspending agents, for example, sodium carboxymethylcellulose, methylcellulose, hydroxypropylmethylcellulose, sodium alginate, polyvinylpyrrolidone, gum tragacanth and gum acacia; dispersing or wetting agents which may be a naturally-occurring phosphatide, for example, lecithin; a condensation product of an alkylene oxide with a fatty acid, for example, polyoxyethylene stearate; a condensation product of ethylene oxide with a long chain aliphatic alcohol, for example, heptadecaethyleneoxycetanol; a condensation product of ethylene oxide with a partial ester derived from fatty acids and a hexitol such as polyoxyethylene sorbitol monooleate; or a condensation product of ethylene oxide with a partial ester derived from fatty acids and hexitol anhydrides, for example, polyoxyethylene sorbitan monooleate. The aqueous suspensions may also contain one or more preservatives, for example, ascorbic acid, ethyl, n-propyl, or p-hydroxybenzoate; or one or more coloring agents.

In one illustrative embodiment, dispersible powders and granules suitable for preparation of an aqueous suspension by the addition of water provide the active ingredient in admixture with a dispersing or wetting agent, suspending agent and one or more preservatives. Additional excipients, for example, coloring agents, may also be present.

Suitable emulsifying agents may be naturally-occurring gums, for example, gum acacia or gum tragacanth; naturally-occurring phosphatides, for example, soybean lecithin; and esters including partial esters derived from fatty acids and hexitol anhydrides, for example, sorbitan mono-oleate, and condensation products of the said partial esters with ethylene oxide, for example, polyoxyethylene sorbitan monooleate.

In other embodiments, isotonic agents, for example, sugars, polyalcohols such as mannitol, sorbitol, or sodium chloride can be included in the composition. Prolonged absorption of the injectable compositions can be brought about by including in the composition an agent which delays absorption, for example, monostearate salts and gelatin.

In one aspect, a conjugate as described herein may be administered directly into the blood stream, into muscle, or into an internal organ. Suitable routes for such parenteral administration include intravenous, intraarterial, intraperitoneal, intrathecal, epidural, intracerebroventricular, intraurethral, intrasternal, intracranial, intratumoral, intramuscular and subcutaneous delivery. Suitable means for parenteral administration include needle (including microneedle) injectors, needle-free injectors and infusion techniques.

In one illustrative aspect, parenteral formulations are typically aqueous solutions which may contain carriers or excipients such as salts, carbohydrates and buffering agents (preferably at a pH of from 3 to 9), but, for some applications, they may be more suitably formulated as a sterile non-aqueous solution or as a dried form to be used in conjunction with a suitable vehicle such as sterile, pyrogen-free water. In other embodiments, any of the liquid formulations described herein may be adapted for parenteral administration of the conjugates described herein. The preparation of parenteral formulations under sterile conditions, for example, by lyophilization under sterile conditions, may readily be accomplished using standard pharmaceutical techniques well known to those skilled in the art.

In one embodiment, the solubility of a conjugate used in the preparation of a parenteral formulation may be increased by the use of appropriate formulation techniques, such as the incorporation of solubility-enhancing agents.

In various embodiments, formulations for parenteral administration may be formulated to be for immediate and/or modified release. In one illustrative aspect, active agents of the invention may be administered in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PGLA). Methods for the preparation of such formulations are generally known to those skilled in the art. In another embodiment, the conjugates described herein or compositions comprising the conjugates may be continuously administered, where appropriate.

In one embodiment, sterile injectable solutions can be prepared by incorporating the active agent in the required amount in an appropriate solvent with one or a combination of ingredients described above, as required, followed by filtered sterilization. Typically, dispersions are prepared by incorporating the active compound into a sterile vehicle which contains a dispersion medium and any additional ingredients from those described above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof.

In one embodiment, the proper fluidity can be maintained, for example, by the use of a coating such as lecithin, by the maintenance of the required particle size in the case of dispersion and by the use of surfactants.

In various embodiments, formulations for parenteral administration may be formulated to be for immediate and/or modified release. Modified release formulations include delayed, sustained, pulsed, controlled, targeted and programmed release formulations.

The activated macrophage-targeted conjugates used for detecting disease states mediated by activated macrophages in accordance with this invention are formed to target and, thus, to concentrate the ligand conjugate at the site of activated macrophage populations (e.g. activated macrophages adhering to the luminal endothelial layer of the plaque or activated macrophages present in the lipid-rich core of the plaque) in the patient being evaluated for atherosclerosis.

In one embodiment of the invention active atherosclerotic plaques comprising activated macrophages are detected in a patient being evaluated for atherosclerosis by administering a conjugate of the formula L-X wherein L comprises a ligand capable of preferentially binding to activated macrophages, compared to resting macrophages, and X comprises a chromophore or a chemical moiety capable of emitting radiation. The inner lining of a patient's blood vessels is thereafter examined with a catheter-based device capable of detecting a localized concentration of the chromophore/chemical moiety conjugated to the ligand bound to activated macrophages, or by an external imaging technique. Any external imaging technique known in the art can be used.

The ligand conjugates are typically administered as a diagnostic composition comprising a ligand conjugate and a pharmaceutically acceptable carrier. The composition is typically formulated for parenteral administration and is administered to the patient in an amount effective to enable detection of the locale of activated macrophages. The nature of the chromophore/chemical moiety component of the ligand conjugate is dictated by the methodology used for catheter-based detection or external imaging of the active atherosclerotic plaques. Thus, for example, the chromophore can comprise a fluorophore, such as fluorescein, (see PCT publication number WO 01/074382, incorporated herein by reference, for a description of a ligand-fluorophore conjugate) or another chromophore such as rhodamine, coumarin, cyanine, HiLyte Fluors, DyLight Fluors, or Alexa Fluors, Texas Red, phycoerythrin, Oregon Green, Cy3, Cy5, Cy7, and the like, an hematoporphyrin, or a derivative thereof, or a Raman enhancing dye or agent, or a long wavelength fluorescent dye with optical properties that allow detection through many layers of tissue. The component of the ligand conjugate used for detection can also be a chemical moiety, such as a chelating moiety and a metal cation, for example, a radionuclide. It should be noted that the method of the present invention can be used for detecting light or radioactivity emitted from ligand conjugates bound both at the surface of atherosclerotic plaques and below the surface.

In another aspect, the chromophore is a fluorescent agent selected from Oregon Green fluorescent agents, including but not limited to Oregon Green 488, Oregon Green 514, and the like, AlexaFluor fluorescent agents, including but not limited to AlexaFluor 488, AlexaFluor 647, and the like, fluorescein, and related analogs, rhodamine fluorescent agents, including but not limited to tetramethylrhodamine, and the like, DyLight fluorescent agents, including but not limited to DyLight 680, and the like, CW 800, Texas Red, phycoerythrin, and others. Illustrative fluorescent agents are shown in the following illustrative general structures:

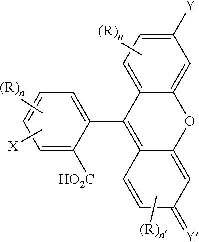

where X is oxygen, nitrogen, sulfur, S(O)₂, or C(O), and where X is attached to linker L; Y is OR^{a}, NR^{a}₂, or NR^{a}₃⁺; and Y′ is O, NR^{a}, or NR^{a}₂⁺; n is in each instance independently selected from 0, 1, 2, or 3; where each R is independently selected in each instance from H, alkyl, alkyloxy, heteroalkyl, fluoro, sulfonic acid, sulfonate, and salts thereof, and the like; and R^{a} is hydrogen, alkly, alkylsulfonic acid, or alkylsulfonate, and salts thereof; or at least one of R and Ra the atoms to which they are attached form a heterocycle; and, in another embodiment,

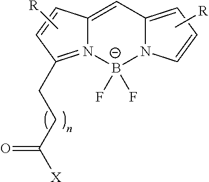

where X is oxygen, nitrogen, or sulfur, and where X is attached to linker L; and each R is independently selected in each instance from H, alkyl, heteroalkyl, and the like; and n is an integer from 0 to about 4; and in another illustrative embodiment,

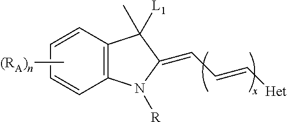

wherein R_{A} and R_{B} are independently selected in each instance from alkyl, heteroalkyl, alkylsulfonic acid, alkylsulfonate, or a salt thereof, or an amine or a derivative thereof; L₁ is a divalent linker attached to the targeting ligand; R is independently selected in each instance from alkyl, heteroalkyl, or alkylsulfonic acid, or alkylsulfonate, or a salt thereof; n is independently in each instance an integer from 0 to about 3; x is an integer from about 1 to about 4; and Het is selected from the group consisting of

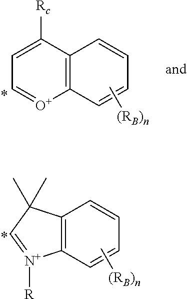

wherein * is the attachment point; and R_{C} is alkyl or heteroalkyl.

The ligand-chromophore conjugate as herein described can be selected, for example, from the group consisting of

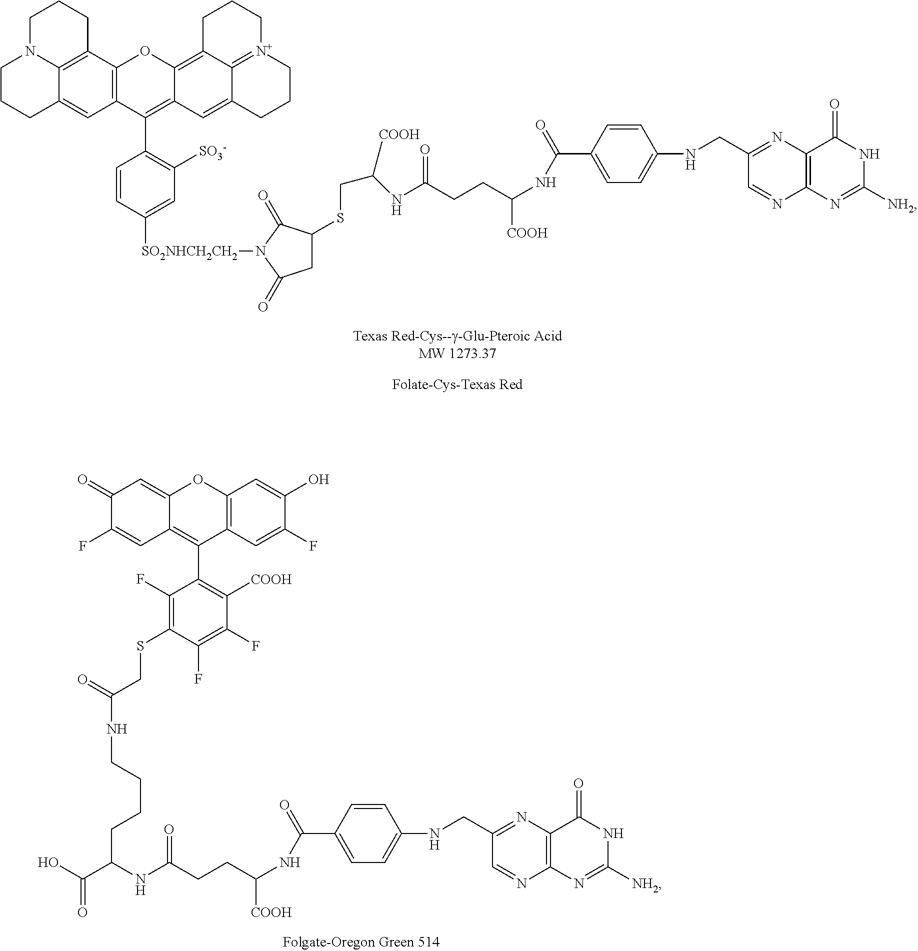

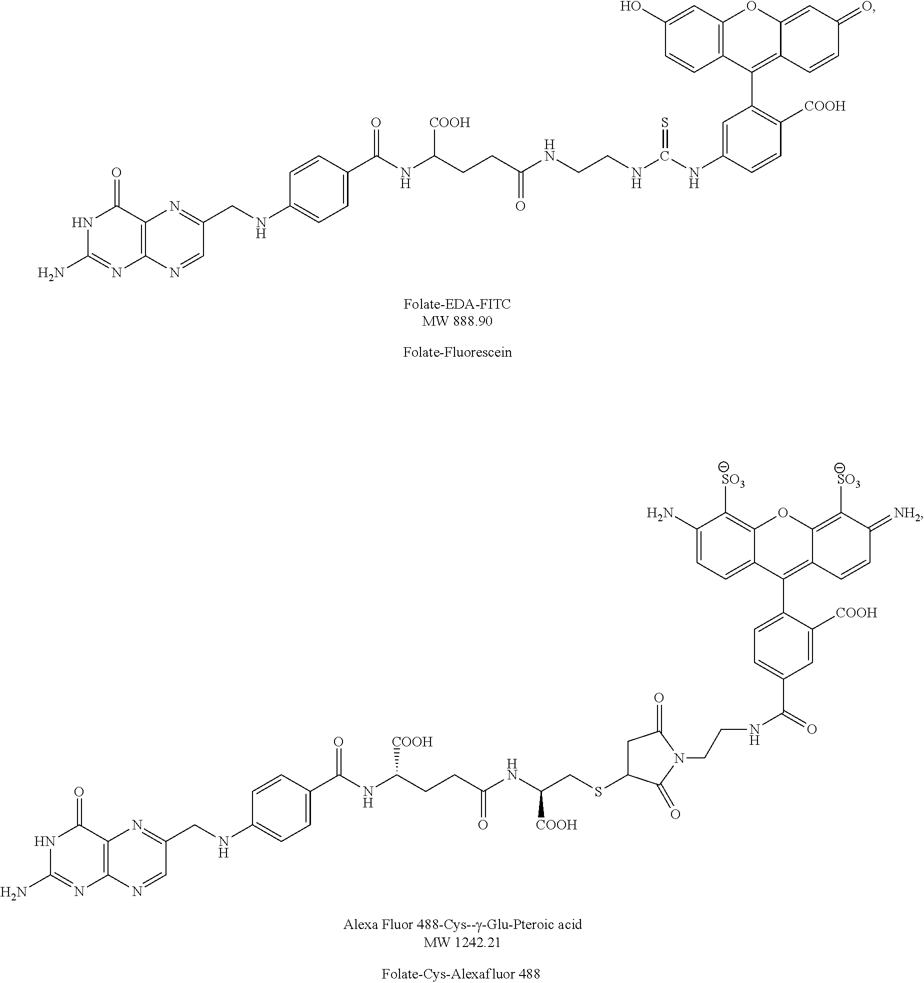

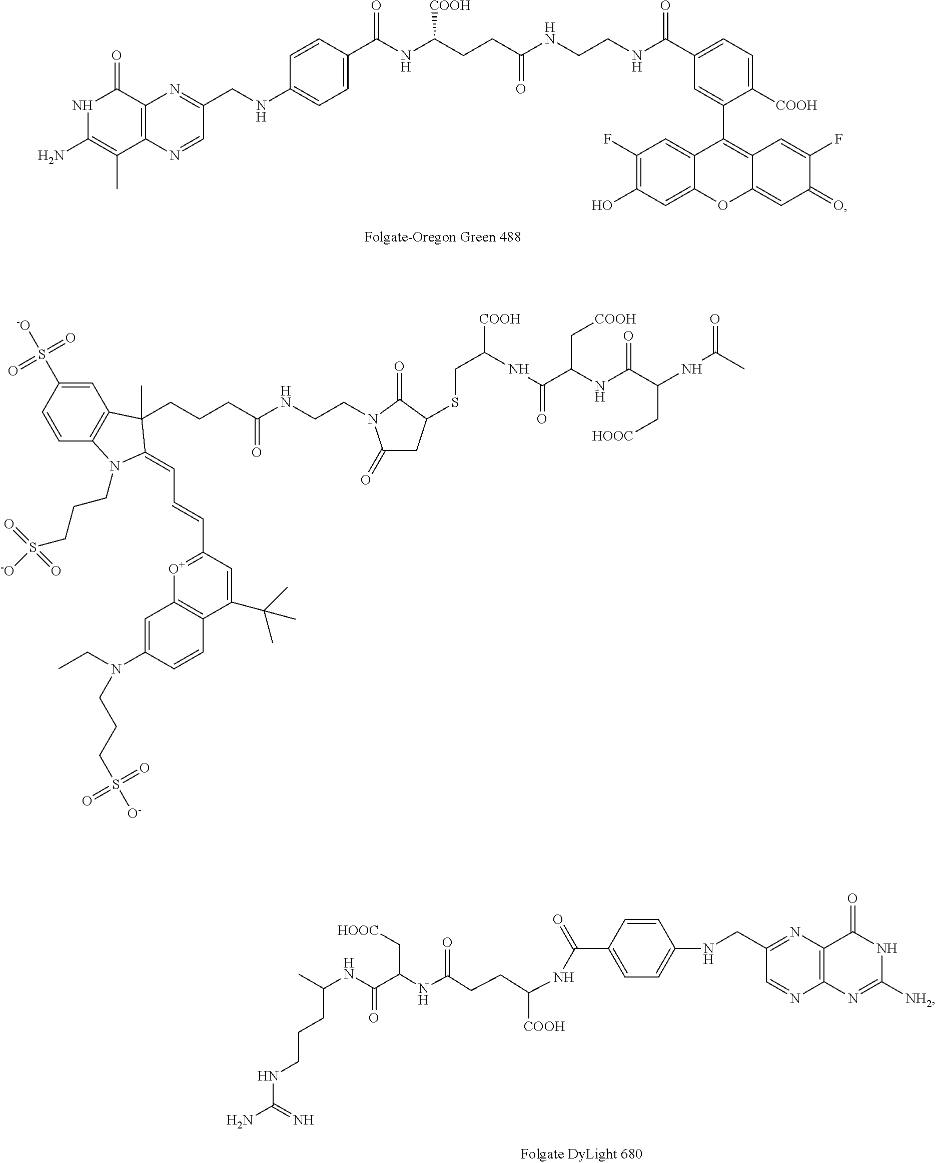

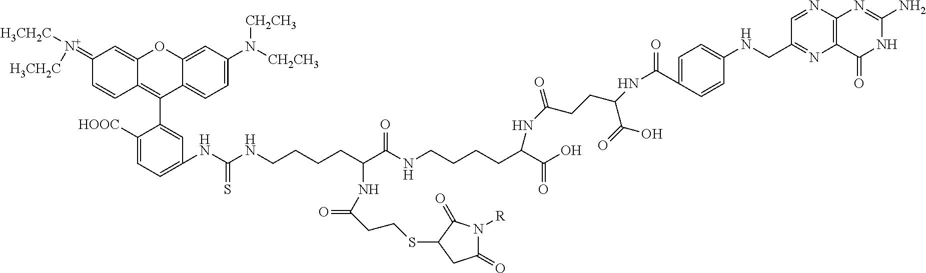

R represents the following:

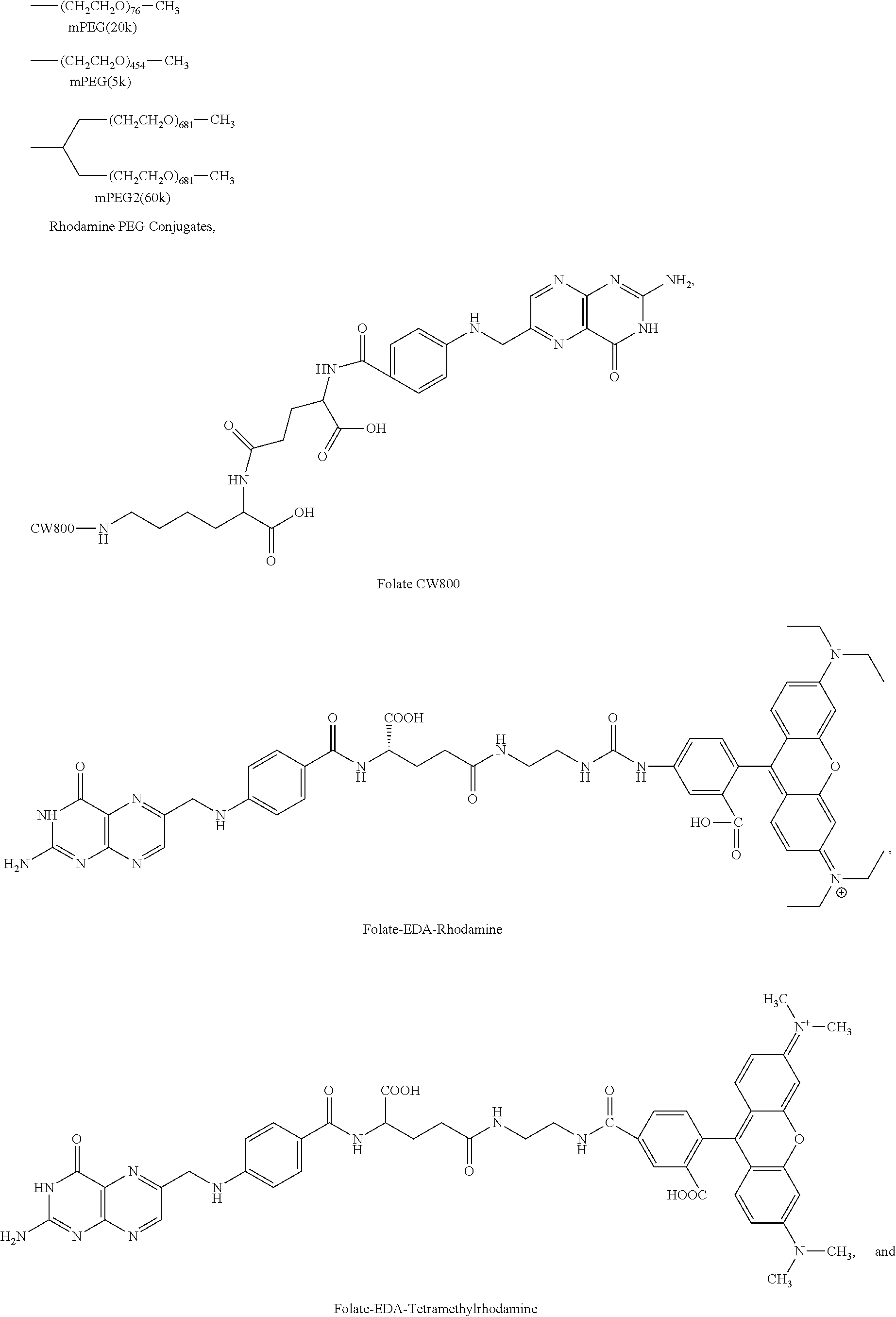

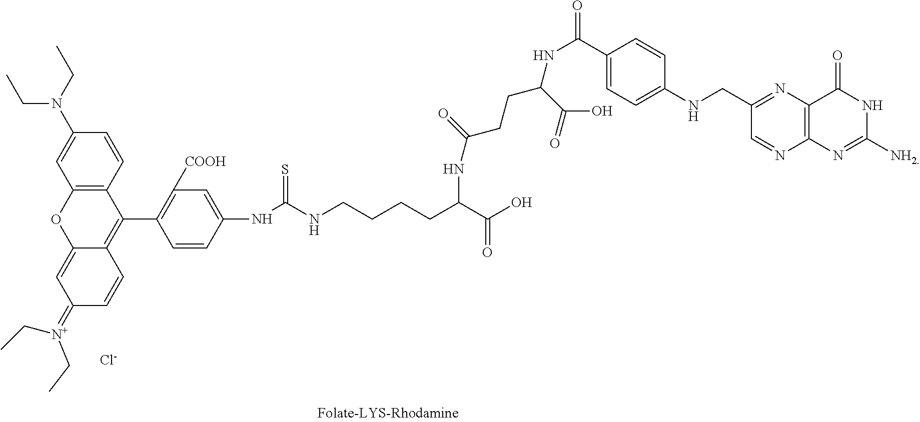

Ligand-chromophore conjugates described herein can be prepared using synthetic procedures described in WO2008/057437, the contents of which are herein incorporated by reference.

Such conjugates wherein the group L is folic acid, a folic acid analog/derivative, or another folic acid receptor binding ligand are described in detail in U.S. Pat. No. 5,688,488, incorporated herein by reference. That patent, as well as related U.S. Pat. Nos. 5,416,016 and 5,108,921, each incorporated herein by reference, describe methods and examples for preparing conjugates useful in accordance with the present invention. The present macrophage-targeted ligand conjugates can be prepared and used following general protocols described in those earlier patents.

In the embodiment where the ligand conjugate comprises a chromophore for use in detecting active atherosclerotic plaques, the blood vessel walls can be subjected to predetermined conditions to detect locations on the inner linings of blood vessels where the ligand-chromophore conjugates are concentrated (i.e., active atherosclerotic plaques). Such predetermined conditions include any conditions known in the art to be useful for the detection of a chromophore, such as a fluorophore, using a catheter-based device or external imaging technique. For example, the blood vessel walls can be subjected to radiation, in the ultraviolet, visible, or infrared region of the spectrum, from a laser. Catheter-based techniques employing optical fibers for the pulsed or steady state illumination of atherosclerotic plaques with laser radiation of a given wavelength can be used. A signal generated by the fluorescent light emitted by the ligand conjugates is then conveyed by one or more of the optical fibers to the end of the catheter where it can be analyzed to yield information about the atherosclerotic plaque being evaluated. The light emitted can be analyzed using art-recognized techniques as described below to identify/monitor the atherosclerotic plaque being evaluated.

In view of the increase in folate receptor levels during macrophage activation, a ligand conjugate comprising a ^{99m}Tc chelating chemical moiety targeted to activated macrophages using a vitamin, such as folate, complexed or chelated to ^{99m}Tc, can be used to detect active plaques in vivo. Such a ligand conjugate, EC20, is described in U.S. Pat. No. 7,128,893, incorporated herein by reference. In one illustrative example, EC20 (^{99m}Tc complex) a ligand conjugate compound of the formula

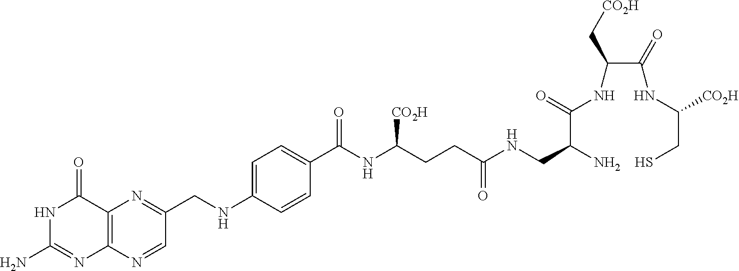

complexed to ^{99m}Tc is used to detect active plaques in vivo.

In another embodiment, dectection of active plaques is accomplished using the ligand conjugate compound of formula

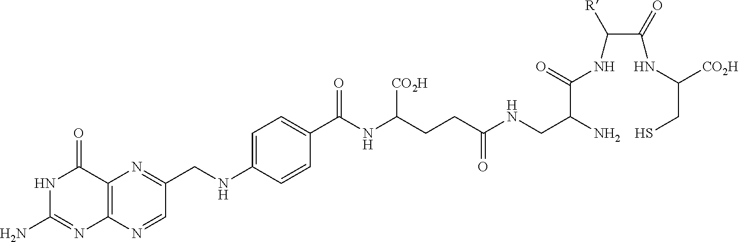

wherein R′ is the side chain of an amino acid.

In another embodiment, detection of active plaques using the ligand conjugate compound of formula

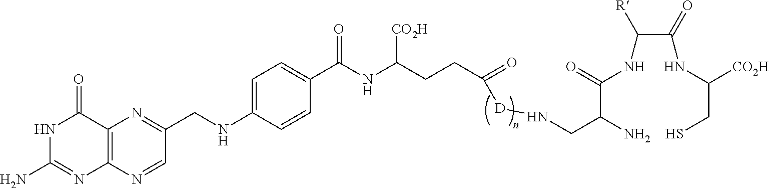

wherein R′ is the side chain of an amino acid, D is a divalent linker, and n is 0 or 1 is described.

In one embodiment, the L-X conjugate (e.g., EC20) is pyrogen-free. In another embodiment, the L-X conjugate (e.g., EC20) is administered after administration of unlabeled folate to the patient.

Typically the activated macrophage-targeted ligand conjugate is administered to a patient, and following a period of time sufficient (e.g., from about 1 to about 24 hours) for the ligand conjugate to bind to activated macrophages associated with the active plaques, the patient is subjected to the catheterization procedure or an external imaging technique and detection of active plaques is enabled by the targeted ligand conjugate.

Active atherosclerotic plaques can be identified/monitored in accordance with the method of the invention by, for example, spectral analysis of fluorescence emitted by the chromophore where the fluorescence emission is stimulated by radiation from, for example, a laser (e.g., laser-induced fluorescence spectroscopy), or by analysis of radioactivity emitted by the chemical moiety. Exemplary analytical techniques are described in U.S. Pats. Nos. 4,718,417 and 4,785,806, and in U.S. Patent Application Publication No. US 2003-0162234 A1, each incorporated herein by reference, but any technique useful for analyzing light or radioactivity emitted from an atherosclerotic plaque to identify/monitor the atherosclerotic plaque in accordance with the invention can be used. In one embodiment, the fluorescence or radioactivity analysis is used to control an ablation laser, and accordingly, the ablation laser is activated, automatically or manually, after the diagnostic laser.

A variety of lasers known in the art can be used in the method of the invention. Exemplary lasers include holmium-doped yttrium aluminum garnet (YAG), holmium-doped yttrium lithium fluoride (YLF), and thulium-doped YAG and thulium-doped YLF. Further details regarding these and other suitable lasers are disclosed in U.S. Pats. Nos. 4,917,084 and 4,950,266, which are hereby incorporated by reference.

The methods described in U.S. Pats. Nos. 5,217,456, 5,275,594, 5,562,100, 6,167,297, 6,217,847, 6,246,901, 6,387,350, 6,507,747, incorporated herein by reference, can also be used to stimulate emission of light from ligand-chromophore conjugates in accordance with the present invention and to detect/analyze light or radioactivity emitted from the ligand conjugates.

The method of the present invention can be used alone or in combination with any other method(s) known in the art for the detection/analysis/ablation of atherosclerotic plaques. For example, the invention can be used in combination with methods to ablate atherosclerotic plaques in cases where active plaques cause narrowing of blood vessels. In such cases, the ligand conjugates of the present invention can be used not only to identify active atherosclerotic plaques as compared to inactive plaques, but also to distinguish between atherosclerotic and normal tissue to help in ablation procedures. Thus, the present invention can be used to analyze both the physiological and the morphological state of atherosclerotic plaques. For example, angioplasty involves the nonsurgical widening of a vessel narrowed by plaque deposition, and laser energy, for example, directed through optical fibers in a catheter-based device, can be used to ablate or partially remove the plaque deposits. Catheter-based devices for ablating plaques using laser energy are described in U.S. Pat. Nos. 4,817,601, 4,850,351, and 4,950,266, incorporated herein by reference.

The method as herein described can be used effectively for detecting atherosclerotic plaques that are small in size. For example, atherosclerotic plaques that result in about 2% occlusion, or blockage, of the lumen of a vessel can be detected using the folate-imaging agent conjugates described herein using a catheter-based device or by external imaging. More particularly, the conjugates described herein can be used to identify/monitor atherosclerotic plaques that block about 2% to about 20%, about 20% to about 50%, about 20% to about 25%, about 25% to about 50%, about 20% to about 30%, about 4% to about 20%, about 4% to about 35%, about 4% to about 10%, about 4% to about 15%, about 2% to about 60%, 4% to about 60% of the lumen of a vessel, about 5% to about 55% of the lumen of a vessel, about 5% to about 50% of the lumen of a vessel, about 2% to about 10% of the lumen of a vessel, about 2% to about 15% of the lumen of a vessel, about 2% to about 25% of the lumen of a vessel, about 2% to about 30% of the lumen of a vessel, or about 2% to about 50% of the lumen of a vessel.

When laser energy is used to ablate an atherosclerotic plaque, thermal damage to normal tissue is a serious risk because the energy level of radiation emitted from lasers used for ablation of plaque can damage or destroy normal tissue with the possibility of inadvertent perforation of an artery. Accordingly, the ligand conjugates of the present invention can be used to not only identify active atherosclerotic plaques, but to distinguish between atherosclerotic plaques and normal tissue to avert damage to normal tissue during plaque ablation. Pulsed laser emission can also be used whenever continuous laser exposure might damage the tissue.

The method of the present invention can also be used in combination with other techniques for differentiating between atherosclerotic plaques (e.g., fibrous plaque, calcified plaque, and lipid plaque) and normal tissue during plaque ablation. Such techniques include techniques based on analysis of laser-induced calcium photoemission from calcified plaque and laser-induced fluorescence from noncalcified plaque. Other such techniques include the analysis of fluorescence (e.g., laser-induced fluorescence), at selected wavelengths from tissues in an artery, with or without the use of a dye to enhance the contrast between the fluorescence emitted from atherosclerotic plaques and the fluorescence emitted from normal tissue (see U.S. Pat. Nos. 4,641,650, 4,718,417, and 4,785,806, incorporated herein by reference). Other laser-based techniques that can be used in combination with the method of the present invention to differentiate between atherosclerotic plaques and normal tissue include techniques utilizing laser-induced Raman light scattering and laser-induced plasma photoemission. Any other type of technique employing diagnostic and/or ablation lasers known in the art can also be used in combination with the method of the present invention (see U.S. Pat. Nos. 4,817,601 and 4,850,351, incorporated herein by reference).

The method of the present invention can also be used in combination with any other method(s) known in the art for the detection/analysis/ablation of atherosclerotic plaques, including the methods described in U.S. Pat. Nos. 5,217,456, 5,275,594, 5,562,100, 6,167,297, 6,217,847, 6,246,901, 6,387,350, 6,507,747, incorporated herein by reference. Furthermore, the invention can be used to guide the positioning of therapeutic drugs and nucleic acid constructs positioned in the same catheter assembly or a different catheter assembly (see U.S. Patent Application Publication No. US 2002-0192157 A1, incorporated herein by reference).

It is also appreciated that in the embodiments described herein, certain aspects of the methods are presented in the alternative, such as selections for any one or more of L or X in the conjugates L-X. It is therefore to be understood that various alternate embodiments of the invention include individual members of those lists, as well as the various subsets of those lists. Each of those combinations are to be understood to be described herein by way of the lists.

The compounds described herein may contain one or more chiral centers, or may otherwise be capable of existing as multiple stereoisomers. Accordingly, it is to be understood that the present invention includes pure stereoisomers as well as mixtures of stereoisomers, such as enantiomers, diastereomers, and enantiomerically or diastereomerically enriched mixtures. The compounds described herein may be capable of existing as geometric isomers. Accordingly, it is to be understood that the present invention includes pure geometric isomers or mixtures of geometric isomers.

As used herein, the term “alkyl” includes a chain of carbon atoms, which is optionally branched. As used herein, the term “alkylene” includes a divalent chain of carbon atoms, which is optionally branched. As used herein, the term “alkenyl” and “alkynyl” includes a chain of carbon atoms, which is optionally branched, and includes at least one double bond or triple bond, respectively. It is to be understood that alkynyl may also include one or more double bonds. It is to be further understood that alkyl is advantageously of limited length, including C₁-C₂₄, C₁-C₁₂, C₁-C₈, C₁-C₆, and C₁-C₄. It is to be further understood that alkenyl and/or alkynyl may each be advantageously of limited length, including C₂-C₂₄, C₂-C₁₂, C₂-C₈, C₂-C₆, and C₂-C₄. It is appreciated herein that shorter alkyl, alkenyl, and/or alkynyl groups may add less lipophilicity to the compound and accordingly will have different pharmacokinetic behavior.

As used herein, the term “heteroalkyl” includes a chain of atoms that includes both carbon and at least one heteroatom, and is optionally branched. Illustrative heteroatoms include nitrogen, oxygen, and sulfur. In certain variations, illustrative heteroatoms also include phosphorus, and selenium.

As used herein, the term “aryl” includes monocyclic and polycyclic aromatic groups, including aromatic carbocyclic and aromatic heterocyclic groups, each of which may be optionally substituted. As used herein, the term “carboaryl” includes aromatic carbocyclic groups, each of which may be optionally substituted. Illustrative aromatic carbocyclic groups described herein include, but are not limited to, phenyl, naphthyl, and the like. As used herein, the term “heteroaryl” includes aromatic heterocyclic groups, each of which may be optionally substituted. Illustrative aromatic heterocyclic groups include, but are not limited to, pyridinyl, pyrimidinyl, pyrazinyl, triazinyl, tetrazinyl, quinolinyl, quinazolinyl, quinoxalinyl, thienyl, pyrazolyl, imidazolyl, oxazolyl, thiazolyl, isoxazolyl, isothiazolyl, oxadiazolyl, thiadiazolyl, triazolyl, benzimidazolyl, benzoxazolyl, benzthiazolyl, benzisoxazolyl, benzisothiazolyl, and the like.

As used herein, the term “amino” includes the group NH₂, alkylamino, and dialkylamino, where the two alkyl groups in dialkylamino may be the same or different, i.e. alkylalkylamino. Illustratively, amino includes methylamino, ethylamino, dimethylamino, methylethylamino, and the like. In addition, it is to be understood that when amino modifies or is modified by another term, such as aminoalkyl, or acylamino, the above variations of the term amino are included therein. Illustratively, aminoalkyl includes H₂N-alkyl, methylaminoalkyl, ethylaminoalkyl, dimethylaminoalkyl, methylethylaminoalkyl, and the like. Illustratively, acylamino includes acylmethylamino, acylethylamino, and the like.

As used herein, the term “amino and derivatives thereof” includes amino as described herein, and alkylamino, alkenylamino, alkynylamino, heteroalkylamino, heteroalkenylamino, heteroalkynylamino, cycloalkylamino, cycloalkenylamino, cycloheteroalkylamino, cycloheteroalkenylamino, arylamino, arylalkylamino, arylalkenylamino, arylalkynylamino, acylamino, and the like, each of which is optionally substituted. The term “amino derivative” also includes urea, carbamate, and the like.

The term “optionally substituted” as used herein includes the replacement of hydrogen atoms with other functional groups on the radical that is optionally substituted. Such other functional groups illustratively include, but are not limited to, amino, hydroxyl, halo, thiol, alkyl, haloalkyl, heteroalkyl, aryl, arylalkyl, arylheteroalkyl, nitro, sulfonic acids and derivatives thereof, carboxylic acids and derivatives thereof, and the like.

While certain embodiments of the present invention have been described and/or exemplified herein, it is contemplated that considerable variation and modification thereof are possible. Accordingly, the present invention is not limited to the particular embodiments described and/or exemplified herein.

### EXAMPLES

### Example 1

### Preparation of EC20-^{99m}Tc

EC20-^{99m}Tc was prepared as described (Leamon et al., Bioconjug Chem, 2002, 13(6): 1200-10; incorporated herein by reference). Vials containing lyophilized EC20 were heated at 100° C. for 5 min, after which two mL of a 925 MBq/mL solution of sodium pertechnetate (Cardinal Health) was added and the vial was heated for an additional 15 min. After dilution with the desired volume of saline, mice were injected i.p. with either 400 μL of imaging agent (18.5 MBq, ˜250 nmoles/Kg of EC20) or the same volume of imaging agent supplemented with 100-fold molar excess of free folic acid (to compete for unoccupied folate receptors). Unbound EC20-^{99m}Tc was allowed to clear from the tissues for a period of four hours prior to imaging.

### Example 2

### Animals—Induction of Atherosclerosis

ApoE−/− breeding trios (Jackson Laboratories) were maintained in a temperature- and humidity-controlled room on a 12 hour dark-light cycle. Female mice were weaned at 3 weeks of age and maintained on either normal rodent chow or transferred at five weeks of age to a Western diet consisting of 2% cholesterol, and 21.2% fat (Harlan-Teklad), as indicated above.

ApoE−/− mice were transferred to a high fat/cholesterol diet (Western Diet) for study—Harlan-Teklad TD.88137. ApoE−/− mice represent a well-known animal model for atherosclerosis. Unless otherwise indicated, mice were kept until 31 weeks on the diet. For example, five week old ApoE−/− mice were transferred and fed the Western Diet for 26 weeks. At different time points after transferring to the Western Diet mice were imaged using a KODAK Imaging Station In Vivo FX.

### Example 3

### Imaging

EC20-^{99m}Tc was prepared as described above. Animals were allowed to clear for a period of 4 hours prior to imaging. Animals were either anesthetized with 3 to 4% isoflurane or euthanized for the imaging procedure. Images were taken in a KODAK Imaging Station In Vivo FX using the following settings. Image acquisition and ROI analyses were performed using KODAK Molecular Imaging software v. 4.5 (Carestream Molecular Imaging).

White Light Imaging:
1. f-stop—22
2. FOV—200×200 mm
3. Emission—White
4. Excitation—Open
5. Exposure time—0.05 seconds
6. Focus—7 mm

Radioimaging:
1. f-stop—0
2. FOV—200×200 mm
3. Emission—Black
4. Excitation—Open
5. Exposure time—60 seconds
6. Focus—7 mm
7. Radioisotopic phosphor screen

X-Ray Imaging:
1. f-stop—4
2. FOV—200×200 mm
3. Emission—Black
4. Excitation—Open
5. Exposure time—55 seconds
6. Focus—7 mm
7. Radiographic phosphor screen

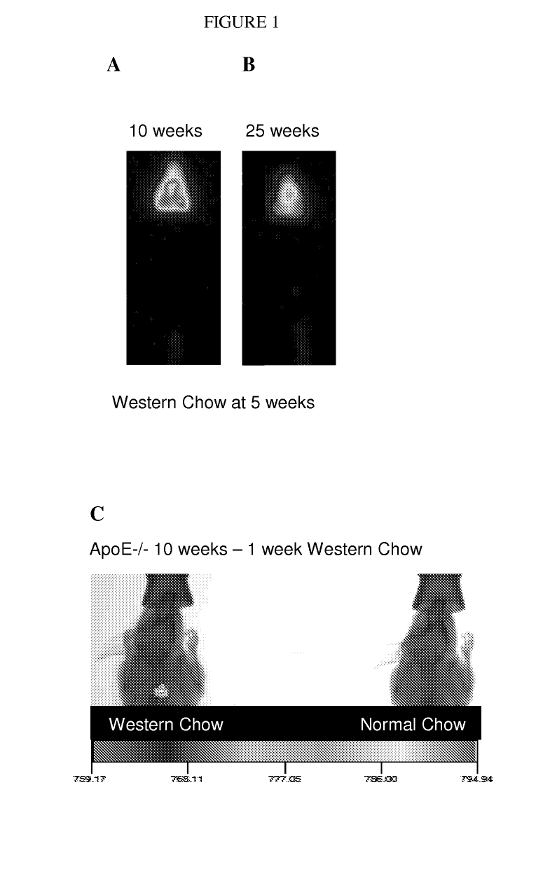

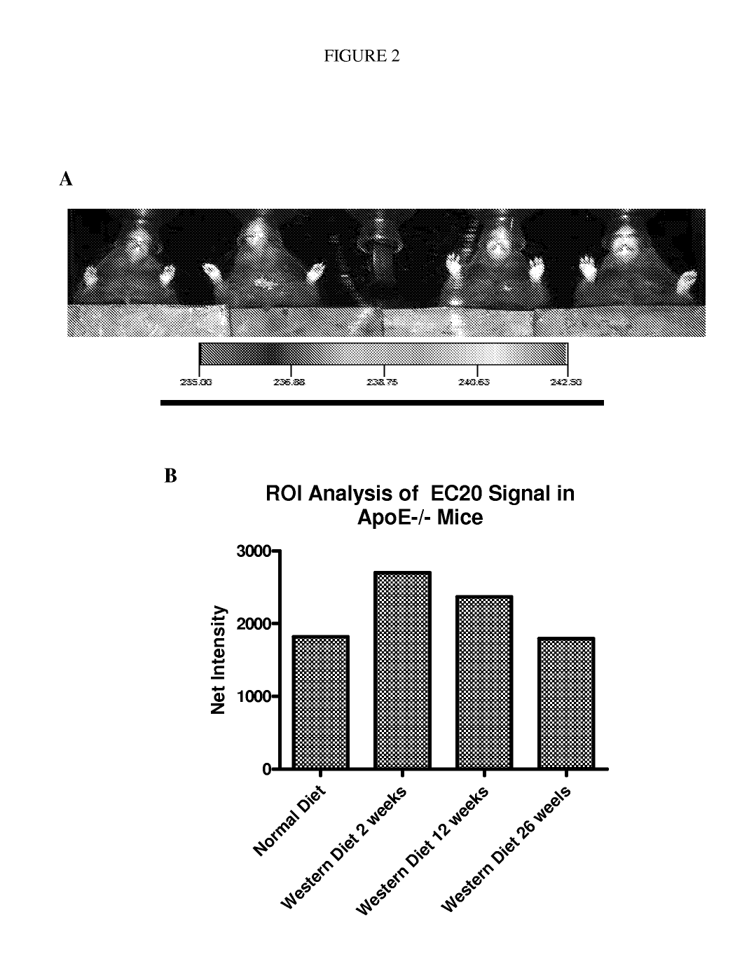

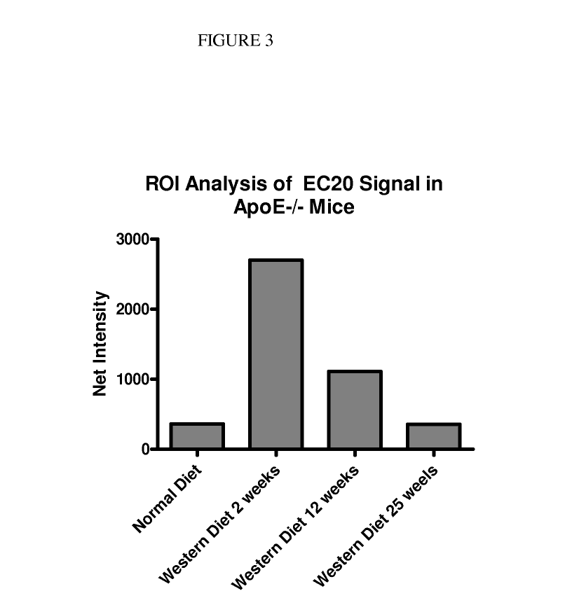

FIGS. 1, 2, and 3 show the early detection of the EC20 signal in ApoE−/− mice fed a high fat/cholesterol diet (Western Diet). FIG. 1 shows ApoE−/− mice fed the Western Diet for 1, 10, or 25 weeks. FIG. 2 shows ApoE−/− mice fed the Western Diet for 0, 2, 12, or 26 weeks. FIG. 3 shows ApoE−/− mice fed the Western Diet for 0, 2, 12, or 25 weeks. The results indicate that EC20 uptake was maximal in small, active atherosclerotic plaques after only 1 or 2 weeks on the high fat Western Diet.Abdomens were shielded with a 5 mm-thick lead shield to mask radioactivities emanating from the kidneys and bladder. Both radiographic and radioimages had a focus setting of 7 mm and a field of view of 200×200 mm. Gamma-scintigraphic images were acquired for 1 minute using a radioisotopic phosphor screen (Carestream Molecular Imaging), no illumination source, 4×4 binning setting, and an f-stop of 0. Radiographic images were acquired for 55 s using a Kodak radiographic phosphor screen (Carestream Molecular Imaging) and used to co-register anatomical structures with radioisotopic signals during overlays. The following settings were employed for X-rays: energy of 35 KVP, current of 149 μA, no X-ray filter, and an f-stop of 4. Signal quantitation was performed using regions of interest analysis. Net intensities were recorded and plotted using Graphpad Prism Software v.4.

To analyze the accumulation of EC20-^{99m}Tc in mouse aortas and heart tissues, mice were euthanized and thoracic aortas excised. Radioactivities were counted for 2 min using a gamma-counter (Packard). Results are reported as % ID/g tissue.

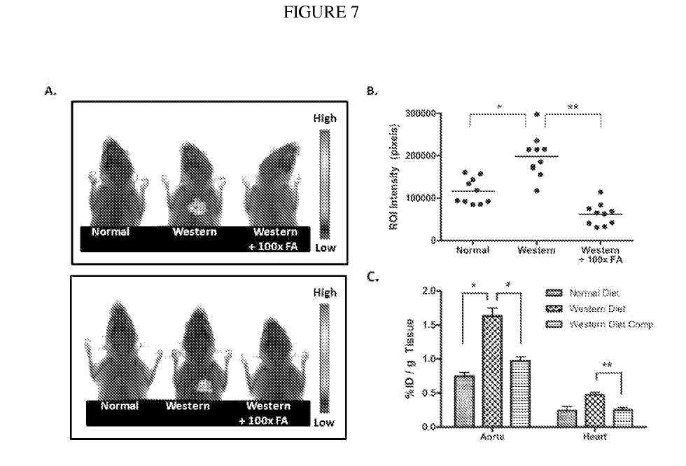

Results indicate that EC20-^{99m}Tc targets the atherosclerotic aortas of apoE−/− mice by binding to the folate receptor. Development of atherosclerosis in apoE−/− mice can be accelerated by maintaining the mice on high fat (Western) diet. To evaluate the ability of EC20-^{99m}Tc to image atherosclerotic lesions, apoE−/− mice were fed either normal or Western chow for 25 weeks, injected i.p. with the above radiopharmaceutical, and then analyzed by radioimaging. As seen in FIG. 7A, apoE−/− mice fed a Western diet exhibited an average increase of ˜70% in EC20-^{99m}Tc signal intensity in the aorto-cardiac region compared to apoE−/− mice maintained on normal rodent chow. When similar atherosclerotic mice on Western diet were pre-injected with 100-fold excess free folic acid to compete with EC20-^{99m}Tc for binding to folate receptors, the signal intensity was reduced to near background levels (FIG. 7B). These data suggest that atherosclerotic lesions are enriched in FR⁺ cells and that uptake of EC20-^{99m}Tc is FR-mediated.

Previous studies have demonstrated that the major sites of atherosclerotic lesion development in apoE−/− mice occur in the aortic root, aortic arch and associated branching arteries. In order to assess whether EC20-^{99m}Tc is in fact targeting these regions of enhanced atherosclerosis, thoracic aortas and hearts were dissected, and accumulation of EC20-^{99m}Tc in the resected tissues was quantitated by gamma counting. As shown in FIG. 7C, EC20-^{99m}Tc uptake was threefold lower in the hearts than in the aortas, and accumulation in the aortas was ˜120% higher in mice on Western chow than normal diet. Moreover, competition with excess folic acid decreased EC20-^{99m}Tc retention in the aortas by 41% compared to non-competed controls (FIG. 7C).

### Example 4

### Autoradiography and Histology

In order to image areas of accumulation of EC20-^{99m}Tc in atherosclerotic aortas, apoE−/− mice on a normal or Western diet were injected with EC20-^{99m}Tc, euthanized, and thoracic aortas were excised. Aortas were excised as described (Martinic et al., Contemp Top Lab Anim Sci 2003, 42(5): 47-53). For cross sections, aortic roots and arches were cut and embedded in Tissue-Tek® O.C.T.™ mounting medium and frozen in liquid nitrogen. Serial sections were cut with a Leica CM1800 cryostat and placed on polylysine coated microscope slides (Thermo Scientific). Either whole aortas or aortic arch cross sections (40 μm) were exposed to a phosphor screen for 18 hours at 4° C. The phosphor screen was read using a Typhoon phosphorimager (GE Healthcare) at a resolution of 50 microns. Aortic tissue sections (10 m thick) adjacent to those used for autoradiography were also used for histology. H&E staining was performed to visualize lesion morphology. H&E staining of the sections was performed as follows.

The slides were fixed for 10 minutes in zinc-buffered formalin. The slides were washed with distilled water. The slides were immersed in Gills-3 hematoxylin for 5 minutes. The slides were rinsed in distilled water and dipped twice in acidic ethanol. The slides were rinsed for 30 seconds with distilled water and for 3 minutes in tap water. The slides were transferred to alcoholic Eosin Y for 20 seconds. The slides were rinsed with water and dehydrated in 2 changes of 95% ethanol, 2 changes of 100% ethanol, and 2 changes of xylenes (3 minutes per change). Coverslips were mounted using Permount™ mounting medium and allowed to dry overnight. Slides were visualized with a light microscope (4× objective). In some cases, the percentage of lumen occlusion was analyzed using ImageJ software (National Institutes of Health).

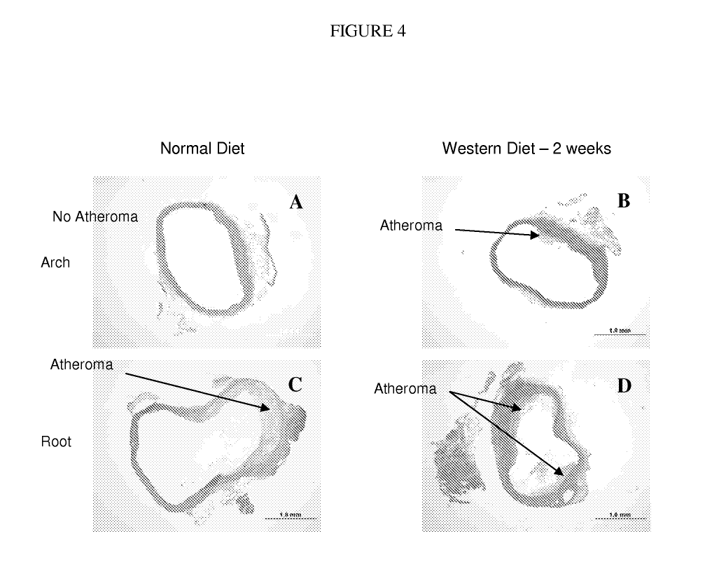

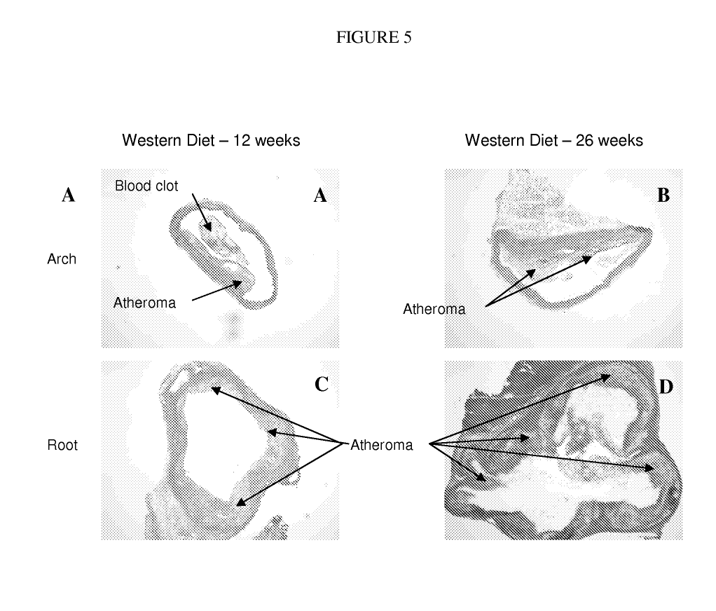

FIGS. 4 and 5 show hematoxylin and eosin (H&E) staining of atherosclerotic plaques in ApoE−/− mice. FIGS. 4 and 5 show the size of the atherosclerotic plaques in ApoE−/− mice versus time on the Western Diet. Specifically, FIG. 4 shows H&E staining of atherosclerotic plaques in ApoE−/− mice fed the Western Diet for 0 or 2 weeks, and FIG. 5 shows H&E staining of atherosclerotic plaques in ApoE−/− mice fed the Western Diet for 12 or 26 weeks. The results show that EC20 uptake is detectable in small, early, active atherosclerotic plaques.

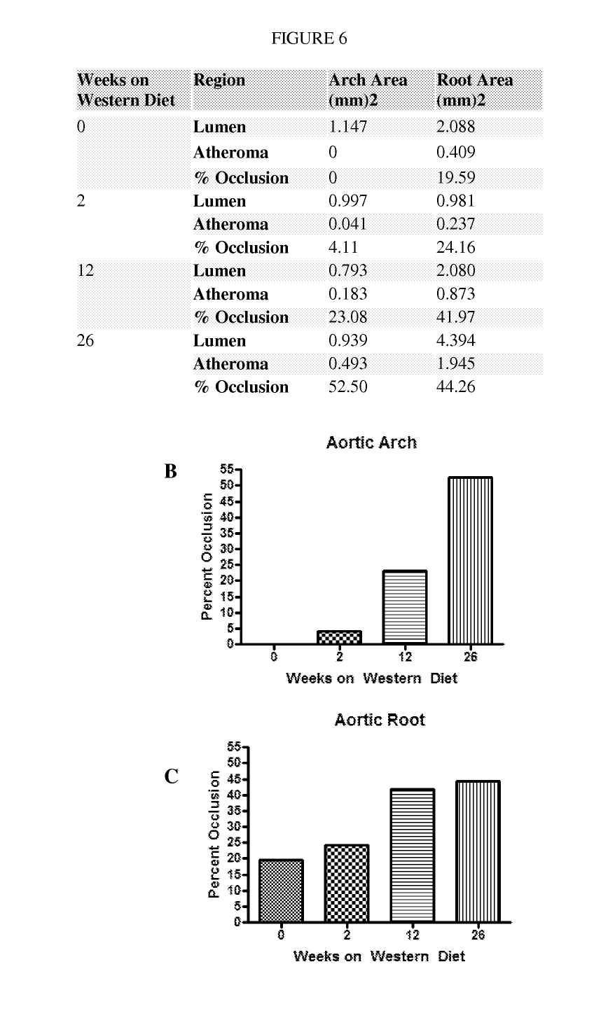

FIG. 6 shows the percent occlusion of the lumen of vessels by atherosclerotic plaques in ApoE−/− mice fed the Western Diet for 2, 12, or 26 weeks. Small active atherosclerotic plaques were detected with a folate-imaging agent conjugate after 2 weeks on the high fat Western Diet. Specifically, atherosclerotic plaques resulting in as little as 4% occlusion of the lumen of the vessel were detected using folate imaging agent conjugates. Percentage of lumen occlusion was determined using ImageJ software following H&E staining.

Staining with the macrophage-specific monoclonal antibody (Mac-3/CD107b; eBioscience Inc.) was performed as follows. Aortic arch sections were fixed with zinc-buffered formalin for 10 min, and endogenous biotin and peroxidase activity were blocked. Sections were incubated with anti-mouse CD107b antibody (1:50 dilution) for 1 h, and after washing, incubated with goat anti-rat biotinylated antibody (KPL Protein Research Products) at a 1:500 dilution for 30 min. After washing, streptavidin-HRP (BD Pharmingen) was added for an additional 30 min. Slides were developed with diaminobenzidine substrate (BD Pharmingen) according to manufacturer's instructions. Negative control consisted of slides developed in the absence of primary antibody. An Olympus BH-2 microscope coupled with a CCD camera was used to obtain light photomicrographs.

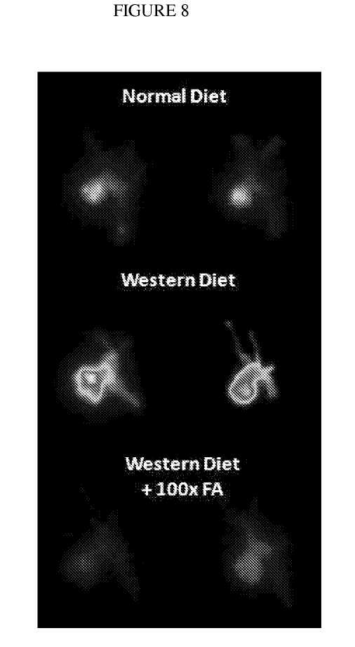

Uptake of EC20-^{99m}Tc in the aortas of a different set of similarly treated apoE−/− mice was examined by autoradiography. As shown in FIG. 8, aortas of mice on the Western diet showed significantly greater uptake in the aortic root and arch than mice fed a normal diet. However, aortas from mice on normal chow also exhibited uptake in their aortic roots, albeit at a lower level; i.e., consistent with the observation that apoE−/− mice spontaneously develop atherosclerotic lesions even on a normal diet. Also, when mice fed the Western diet were administered a 100-fold greater dose of free folic acid than ^{99m}Tc-EC20, the radioactivity in the aortic root and arch was significantly reduced (FIG. 8), suggesting again that uptake was FR-mediated.

### Example 5

### Synthesis and Treatment Using Clodronate Liposomes

PBS- and clodronate liposomes were synthesized as described (Buiting et al., J. Immunol. Methods, 1996, 192(1-2): 55-62; incorporated herein by reference). 86 mg egg phosphatidylcholine +8 mg cholesterol were dissolved in 1:1 chloroform:methanol. Solvent was evaporated using a rotoevaporator for 15 min, and the resulting film was rehydrated with PBS or a 0.6 M solution of clodronate (Sigma) in PBS for 2 hours. Resulting multi-lamellar vesicles were sonicated for 3 min and allowed to swell for 2 hours at 25° C. Liposomes were washed 3× with PBS by centrifugation at 100,000×g for 30 min and resuspended in 4 mL PBS. Liposomes were extruded 5× through both a 400 nm and 200 nm pore-size polycarbonate filter and stored at 4° C. until use. The resulting liposomes consisted of 7:1.3 molar ratio egg phosphatidylcholine:cholesterol, respectively. The efficiency of clodronate entrapment using this method was 7.8%.

For systemic elimination of macrophages, apoE−/− mice were fed a Western diet for a period of 8 weeks, after which 200 μL of PBS- or clodronate-liposomes (4 mg clodronate/dose) were injected i.p. daily for 5 days. After treatment, mice were injected i.p. with EC20-^{99m}Tc and imaged, as described above.

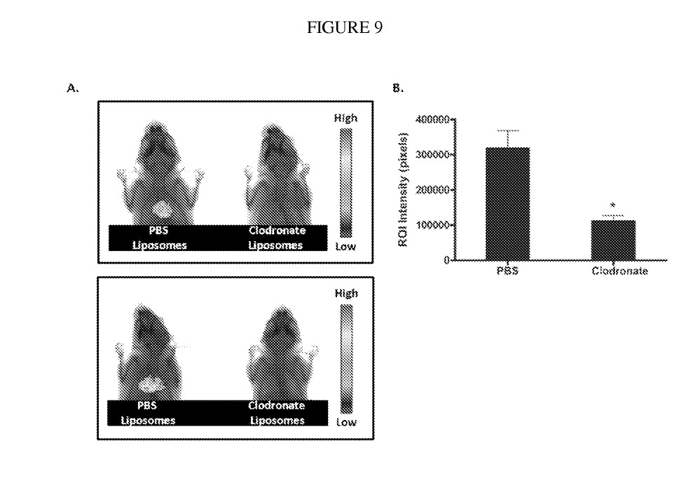

ApoE−/− mice maintained on a Western diet for 8 weeks were treated with clodronate liposomes to systemically eliminate macrophages. After macrophage depletion, mice were injected with EC20-^{99m}Tc and imaged. As shown in FIG. 9, clodronate-liposome treatment reduced uptake of EC20-^{99m}Tc by 65% relative to mice injected with analogous PBS-containing liposomes. These data suggest that FR⁺ macrophages are primarily responsible for uptake of EC20-^{99m}Tc in the atherosclerotic lesions. The data are also consistent with the role of FR⁺ macrophages in mediating uptake of EC20-^{99m}Tc in rheumatoid arthritic and osteoarthritic joints.

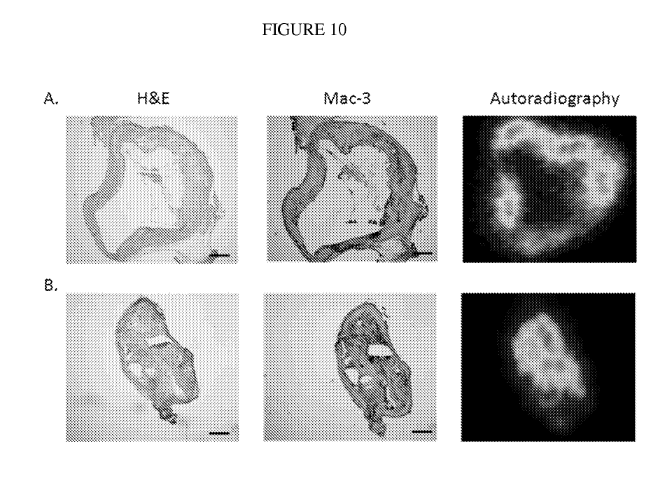

To further establish that macrophages are responsible for accumulation of EC20-^{99m}Tc in atherosclerotic lesions, apoE−/− mice fed for 25 weeks on Western chow were injected with EC20-^{99m}Tc and their aortas examined by autoradiography and histochemistry. For this purpose, the aforementioned mice were euthanized 4 h after i.p. injection of EC20-^{99m}Tc and aortas were resected and cryosectioned, as described above. To compare loci of enhanced macrophage accumulation with regions of elevated ^{99m}Tc signal intensity, serial sections were processed as needed for imaging of each of the above variables and then serial sections were compared. Thus, consecutive sections were: i) stained with H&E to reveal vascular morphology, ii) labeled with Mac-3/CD107b to localize sites of macrophage enrichment, and iii) imaged by autoradiography to identify locations of EC20-^{99m}Tc accumulation. As seen in FIG. 10, areas of high macrophage content and atherosclerotic lesion formation invariably corresponded with loci of elevated ^{99m}Tc emission.

### Example 6

### Digestion of Aortas and Flow Cytometry

ApoE−/− mice on a normal or Western diet for 25 weeks were euthanized and their thoracic aortas were dissected. Aortas were transferred to folate deficient RPMI1640 (Invitrogen) containing 12.5% FBS, 1% PS, 1 mg/mL of collagenase type II (Sigma) and 1 mg/mL of elastase type IV (Sigma). Aortas were incubated for a period of 2 h at 37° C. with gentle swirling of the suspension every 30 min. Cells were washed 3× with fresh folate deficient RPMI1640 and resuspended in the same medium in preparation for flow cytometric analyses.

Resulting cell suspensions were incubated for 1 h at 37° C. in a 1:50 dilution of polyclonal rabbit anti-FR antibody (FL-257, Santa Cruz Biotechnologies). After washing, a 1:100 dilution of FITC-conjugated anti-rabbit antibody (Sigma) and a 1:100 dilution of tri-color anti-F4/80 monoclonal antibody (eBioscience) were added and incubated for an additional hour at 37° C. Cells were washed, resuspended in PBS and analyzed in a FACSCalibur flow cytometer (BD Bioscience). All cell analyses were performed using CellQuant software v3.5 (BD Biosciences).

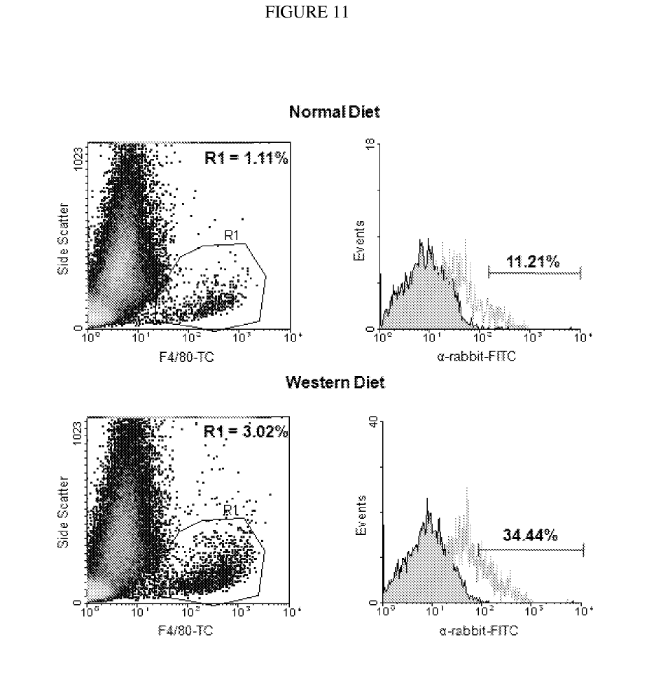

To confirm by yet another method that FR⁺ macrophages play a role in mediating accumulation of EC20-^{99m}Tc in the aortas of apoE−/− mice, thoracic aortas were digested with a cocktail of collagenase and elastase to obtain single cell suspensions, and cells expressing a macrophage marker (F4/80) were analyzed by flow cytometry for simultaneous expression of FR, as described above. As seen in FIG. 11, F4/80⁺ macrophages were found to comprise 1.1% and 3.0% of all cells in the thoracic aortas of mice fed a normal diet and Western diet, respectively. This diet-dependent increase in macrophage content was not unexpected, since an increase in monocyte/macrophage infiltration has been established to constitute a hallmark of atherogenesis. In addition, macrophages from mice fed a normal diet were only 11% FR⁺, whereas macrophages from mice on the Western diet were 33% FR⁺, suggesting that the high fat diet not only increased total macrophage content, but also tripled the percent of FR+ macrophages (FIG. 11). Given that FR expression constitutes a marker for macrophage activation, these data suggest that the higher fat diet elevates both the number and activation state of plaque macrophages.

### Example 7

### Statistical Analysis

Statistical significance among experimental groups was calculated using t-tests. Values of p<0.05 were considered significant.

1. A method of detecting active atherosclerotic plaques wherein the plaques comprise activated macrophages having accessible binding sites for a ligand, and wherein the plaques block from about 2% to about 20% of the lumen of a blood vessel, said method comprising the steps of:
administering to a patient being evaluated for atherosclerosis an effective amount of a composition comprising a conjugate of the general formula
L-X
wherein the group L comprises the ligand and wherein the ligand is a folate, and the group X comprises a chromophore capable of emitting light;
allowing sufficient time for the ligand conjugate to bind to activated macrophages associated with the active plaques; and
detecting active plaques by detecting light emitted by the chromophore using a catheter-based device or by external imaging, wherein the plaques block from about 2% to about 20% of the lumen of a blood vessel.

2. (canceled)

3. The method of claim 1 wherein the chromophore is a fluorophore.

4. The method of claim 3 wherein the fluorophore is selected from the group consisting of a fluorescein, a rhodamine, a cyanine, a DyLight Fluor, and an Alexa Fluor.

5. The method of claim 3 wherein the fluorophore has the formula 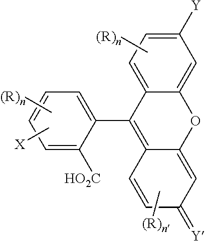
where X is oxygen, nitrogen, sulfur, S(O)₂, or C(O), and where X is attached via a divalent linker to the ligand; Y is OR^{a}, NR^{a}₂, or NR^{a}₃⁺; and Y′ is O, NR^{a}, or NR^{a}₂⁺; n is in each instance independently selected from 0, 1, 2, or 3; where each R is independently selected in each instance from H, alkyl, alkyloxy, heteroalkyl, fluoro, sulfonic acid, sulfonate, and salts thereof; and R^{a} is hydrogen, alkly, alkylsulfonic acid, or alkylsulfonate, and salts thereof; or at least one of R and Ra the atoms to which they are attached form a heterocycle.

6. The method of claim 3 wherein the fluorophore has the formula 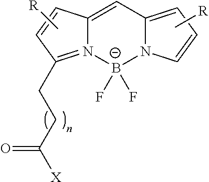
where X is oxygen, nitrogen, or sulfur, and where X is attached via a divalent linker to the ligand; and each R is independently selected in each instance from hydrogen, alkyl, heteroalkyl; and n is an integer from 0 to about 4.

7. The method of claim 3 wherein the fluorophore has the formula 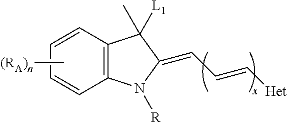
wherein R_{A} and R_{B} are independently selected in each instance from alkyl, heteroalkyl, alkylsulfonic acid, alkylsulfonate, or a salt thereof, or an amine or a derivative thereof; L₁ is an alkylene linked via a divalent linker to the ligand; R is independently selected in each instance from alkyl, heteroalkyl, or alkylsulfonic acid, or alkylsulfonate, or a salt thereof; n is independently in each instance an integer from 0 to about 3; x is an integer from about 1 to about 4; and Het is selected from the group consisting of 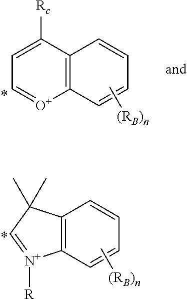
wherein * is the attachment point; and R_{C} is alkyl or heteroalkyl.

8-13. (canceled)

14. The method of claim 1 wherein the folate has the formula 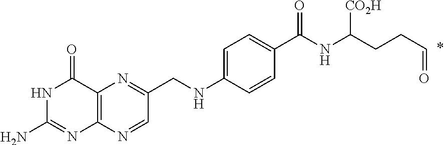
wherein * indicates the attachment point to a divalent linker that links the chromophore and the folate.

15. A method of detecting active atherosclerotic plaques associated with blood vessel walls wherein the plaques comprise activated macrophages having accessible binding sites for a ligand, and wherein the plaques block from about 2% to about 20% of the lumen of a blood vessel, said method comprising the steps of:
administering to a patient suffering from atherosclerosis an effective amount of a composition comprising a conjugate of the general formula
L-X
wherein the group L comprises the ligand and wherein the ligand is a folate, and the group X comprises a chemical moiety capable of emitting radiation;
allowing sufficient time for the ligand conjugate to bind to the activated macrophages associated with the active plaques; and
detecting active plaques by detecting radiation emitted by the chemical moiety using a catheter-based device or by external imaging, wherein the plaques block from about 2% to about 20% of the lumen of a blood vessel.

16. The method of claim 15 wherein the chemical moiety comprises a metal chelating moiety.

17. The method of claim 16 wherein the chemical moiety further comprises a metal cation.

18. The method of 17 wherein the metal cation is a radionuclide.

19. The method of claim 18 wherein the radionuclide is ^{99m}Tc.

20-21. (canceled)

22. The method of claim 15 wherein the conjugate comprises a compound of the formula 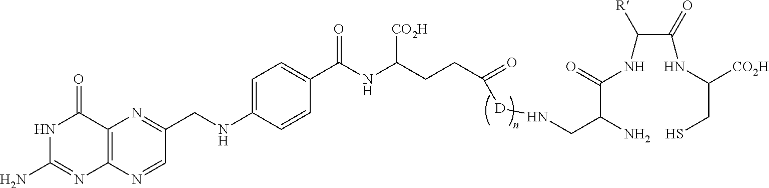
wherein R′ is hydrogen, or R′ selected from the group consisting of alkyl, aminoalkyl, carboxyalkyl, hydroxyalkyl, heteroalkyl, aryl, arylalkyl and heteroarylalkyl, each of which is optionally substituted; D is a divalent linker, n is 0 or 1, and wherein the compound has a bound radionuclide.

23. The method of claim 15 wherein the conjugate comprises a compound of the formula 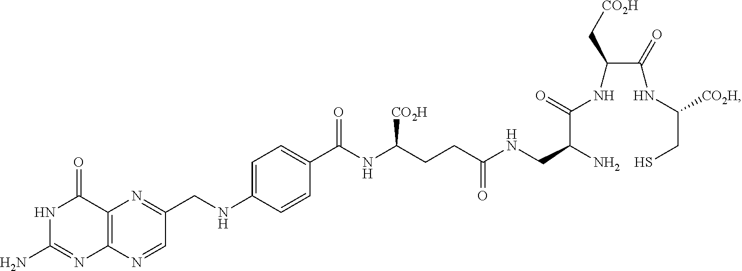
wherein the compound has a bound radionuclide, and wherein the radionuclide is ^{99m}Tc.

24-52. (canceled)

53. The method of claim 15 wherein the folate has the formula 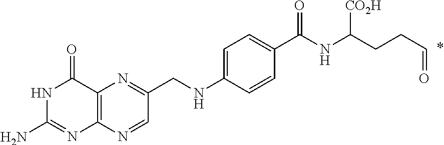
wherein * indicates the attachment point to a divalent linker that links the chromophore and the folate.

54. The method of claim 1 wherein the light emitted by the chromophore is detected using a catheter-based device.

55. The method of claim 1 wherein the light emitted by the chromophore is detected using external imaging.

56. The method of claim 15 wherein the radiation emitted by the chemical moiety is detected using a catheter-based device.

57. The method of claim 15 wherein the radiation emitted by the chemical moiety is detected using external imaging.

58. The method of claim 1 wherein the conjugate has the formula [chemical structure]
